SEQUENCE LISTING (49 sequences)

SEQ ID NO: 1 - protein, 653 aa, homo sapiens
  IDUA polypeptide sequence
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 2 - protein, 653 aa, homo sapiens; identical to SEQ ID NO: 1
  IDS polypeptide sequence

SEQ ID NO: 3 - protein, 533 aa, homo sapiens
  ARSB polypeptide sequence
MGPRGAASLPRGPGPRRLLLPVVLPLLLLLLLAPPGSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 4 - protein, 651 aa, homo sapiens
  GUSB polypeptide sequence
MARGSAVAWAALGPLLWGCALGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSLFT

SEQ ID NO: 5 - protein, 745 aa, homo sapiens [sequence omitted]

SEQ ID NO: 6 - protein, 685 aa, homo sapiens
  GALC  polypeptide sequence
MAEWLLSASWQRRAKAMTAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFTSGMLNDKSLWTDIPVNFPKNGWAAIGTHSFEFAQFDNFLVEATR

SEQ ID NO: 7 - protein, 509 aa, homo sapiens
  ARSA  polypeptide sequence
MSMGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 8 - protein, 527 aa, homo sapiens
  PSAP polypeptide sequence
MYALFLLASLLGAALAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQDQQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 9 - protein, 536 aa, homo sapiens
  GBA polypeptide sequence
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 10 - protein, 466 aa, homo sapiens
  FUCA1 polypeptide sequence
MRAPGMRSRPAGPALLLLLLFLGAAESVRRAQPPRRYTPDWPSLDSRPLPAWFDEAKFGVFIHWGVFSVPAWGSEWFWWHWQGEGRPQYQRFMRDNYPPGFSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQHFVSAKTMPELYDLVNSYKPDLIWSDGEWECPDTYWNSTNFLSWLYNDSPVKDEVVVNDRWGQNCSCHHGGYYNCEDKFKPQSLPDHKWEMCTSIDKFSWGYRRDMALSDVTEESEIISELVQTVSLGGNYLLNIGPTKDGLIVPIFQERLLAVGKWLSINGEAIYASKPWRVQWEKNTTSVWYTSKGSAVYAIFLHWPENGVLNLESPITTSTTKITMLGIQGDLKWSTDPDKGLFISLPQLPPSAVPAEFAWTIKLTGVK

SEQ ID NO: 11 - protein, 1011 aa, homo sapiens
  MAN2B1 polypeptide sequence
MGAYARASGVCARGCLDSAGPWTMSRALRPPLPPLCFFLLLLAAAGARAGGYETCPTVQPNMLNVHLLPHTHDDVGWLKTVDQYFYGIKNDIQHAGVQYILDSVISALLADPTRRFIYVEIAFFSRWWHQQTNATQEVVRDLVRQGRLEFANGGWVMNDEAATHYGAIVDQMTLGLRFLEDTFGNDGRPRVAWHIDPFGHSREQASLFAQMGFDGFFFGRLDYQDKWVRMQKLEMEQVWRASTSLKPPTADLFTGVLPNGYNPPRNLCWDVLCVDQPLVEDPRSPEYNAKELVDYFLNVATAQGRYYRTNHTVMTMGSDFQYENANMWFKNLDKLIRLVNAQQAKGSSVHVLYSTPACYLWELNKANLTWSVKHDDFFPYADGPHQFWTGYFSSRPALKRYERLSYNFLQVCNQLEALVGLAANVGPYGSGDSAPLNEAMAVLQHHDAVSGTSRQHVANDYARQLAAGWGPCEVLLSNALARLRGFKDHFTFCQQLNISICPLSQTAARFQVIVYNPLGRKVNWMVRLPVSEGVFVVKDPNGRTVPSDVVIFPSSDSQAHPPELLFSASLPALGFSTYSVAQVPRWKPQARAPQPIPRRSWSPALTIENEHIRATFDPDTGLLMEIMNMNQQLLLPVRQTFFWYNASIGDNESDQASGAYIFRPNQQKPLPVSRWAQIHLVKTPLVQEVHQNFSAWCSQVVRLYPGQRHLELEWSVGPIPVGDTWGKEVISRFDTPLETKGRFYTDSNGREILERRRDYRPTWKLNQTEPVAGNYYPVNTRIYITDGNMQLTVLTDRSQGGSSLRDGSLELMVHRRLLKDDGRGVSEPLMENGSGAWVRGRHLVLLDTAQAAAAGHRLLAEQEVLAPQVVLAPGGGAAYNLGAPPRTQFSGLRRDLPPSVHLLTLASWGPEMVLLRLEHQFAVGEDSGRNLSAPVTLNLRDLFSTFTITRLQETTLVANQLREAASRLKWTTNTGPTPHQTPYQLDPANITLEPMEIRTFLASVQWKEVDG

SEQ ID NO: 12 - protein, 346 aa, homo sapiens
  AGA polypeptide sequence
MARKSNLPVLLVPFLLCQALVRCSSPLPLVVNTWPFKNATEAAWRALASGGSALDAVESGCAMCEREQCDGSVGFGGSPDELGETTLDAMIMDGTTMDVGAVGDLRRIKNAIGVARKVLEHTTHTLLVGESATTFAQSMGFINEDLSTTASQALHSDWLARNCQPNYWRNVIPDPSKYCGPYKPPGILKQDIPIHKETEDDRGHDTIGMVVIHKTGHIAAGTSTNGIKFKIHGRVGDSPIPGAGAYADDTAGAAAATGNGDILMRFLPSYQAVEYMRRGEDPTIACQKVISRIQKHFPEFFGAVICANVTGSYGAACNKLSTFTQFSFMVYNSEKNQPTEEKVDCI

SEQ ID NO: 13 - protein, 411 aa, homo sapiens
  ASAH1 polypeptide sequence
MNCCIGLGEKARGSHRASYPSLSALFTEASILGFGSFAVKAQWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 14 - protein, 529 aa, homo sapiens
  HEXA polypeptide sequence
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 15 - protein, 952 aa, homo sapiens
  GAA polypeptide sequence
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 16 - protein, 631 aa, homo sapiens
  SMPD1 polypeptide sequence
MPRYGASLRQSCPRSGREQGQDGTAGAPGLLWMGLVLALALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 17 - protein, 399 aa, homo sapiens
  LIPA polypeptide sequence
MKMRFLGLVVCLVLWTLHSEGSGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 18 - protein, 1030 aa, homo sapiens
  CDKL5 polypeptide sequence
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPDGGCDGRRQRHHSGPQDRRFMLRTTEQQGEYFCCGDPKKPHTPCVPNRALHRPISSPAPYPVLQVRGTSMCPTLQVRGTDAFSCPTQQSGFSFFVRHVMREALIHRAQVNQAALLTYHENAALTGK

SEQ ID NO: 19 - protein, 147 aa, homo sapiens
  HBB
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 20 - protein, 261 aa, homo sapiens
  CD40L
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 21 - protein, 1144 aa, homo sapiens
  ADCY3
MPRNQGFSEPEYSAEYSAEYSVSLPSDPDRGVGRTHEISVRNSGSCLCLPRFMRLTFVPESLENLYQTYFKRQRHETLLVLVVFAALFDCYVVVMCAVVFSSDKLASLAVAGIGLVLDIILFVLCKKGLLPDRVTRRVLPYVLWLLITAQIFSYLGLNFARAHAASDTVGWQVFFVFSFFITLPLSLSPIVIISVVSCVVHTLVLGVTVAQQQQEELKGMQLLREILANVFLYLCAIAVGIMSYYMADRKHRKAFLEARQSLEVKMNLEEQSQQQENLMLSILPKHVADEMLKDMKKDESQKDQQQFNTMYMYRHENVSILFADIVGFTQLSSACSAQELVKLLNELFARFDKLAAKYHQLRIKILGDCYYCICGLPDYREDHAVCSILMGLAMVEAISYVREKTKTGVDMRVGVHTGTVLGGVLGQKRWQYDVWSTDVTVANKMEAGGIPGRVHISQSTMDCLKGEFDVEPGDGGSRCDYLEEKGIETYLIIASKPEVKKTATQNGLNGSALPNGAPASSKSSSPALIETKEPNGSAHSSGSTSEKPEEQDAQADNPSFPNPRRRLRLQDLADRVVDASEDEHELNQLLNEALLERESAQVVKKRNTFLLSMRFMDPEMETRYSVEKEKQSGAAFSCSCVVLLCTALVEILIDPWLMTNYVTFMVGEILLLILTICSLAAIFPRAFPKKLVAFSTWIDRTRWARNTWAMLAIFILVMANVVDMLSCLQYYTGPSNATAGMETEGSCLENPKYYNYVAVLSLIATIMLVQVSHMVKLTLMLLVAGAVATINLYAWRPVFDEYDHKRFREHDLPMVALEQMQGFNPGLNGTDRLPLVPSKYSMTVMVFLMMLSFYYFSRHVEKLARTLFLWKIEVHDQKERVYEMRRWNEALVTNMLPEHVARHFLGSKKRDEELYSQTYDEIGVMFASLPNFADFYTEESINNGGIECLRFLNEIISDFDSLLDNPKFRVITKIKTIGSTYMAASGVTPDVNTNGFASSNKEDKSERERWQHLADLADFALAMKDTLTNINNQSFNNFMLRIGMNKGGVLAGVIGARKPHYDIWGNTVNVASRMESTGVMGNIQVVEETQVILREYGFRFVRRGPIFVKGKGELLTFFLKGRDKLATFPNGPSVTLPHQVVDNS

SEQ ID NO: 22 - protein, 247 aa, homo sapiens
  BDNF
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 23 - protein, 950 aa, homo sapiens [sequence omitted]

SEQ ID NO: 24 - protein, 167 aa, homo sapiens
  LEP
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 25 - DNA, 49 bp, artificial
  TRAC TALEN target
ttcctcctactcaccatcagcctcctggttatggtacaggtaagagcaa

SEQ ID NO: 26 - DNA, 47 bp, artificial
  B2M TALEN target
tccgtggccttagctgtgctcgcgctactctctctttctggcctgga

SEQ ID NO: 27 - DNA, 2282 bp, artificial
  HLA-E  AAV insert
caccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactctctcttagcggcctcgaagctgttatggctccgcggactttaattttaggtggtggcggatccggtggtggcggttctggtggtggcggctccatccagcgtacgcccaaaattcaagtctacagccgacatcctgcagagaacggcaaatctaatttcctgaactgctatgtatcaggctttcaccctagcgatatagaagtggacctgctgaaaaacggagagaggatagaaaaggtcgaacacagcgacctctccttttccaaggactggagcttttatcttctgtattatactgaatttacacccacggaaaaagatgagtatgcgtgccgagtaaaccacgtcacgctgtcacagcccaaaatagtaaaatgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgtctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggaca

SEQ ID NO: 28 - DNA, 2572 bp, artificial
  Mesothelin CAR AAV insert
gataagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagggcagcggcgtgaagcagaccctgaacttcgacctgctgaagctggcaggcgatgtggagtccaatccaggacctatggctctgcccgtcaccgctctgctgctgccactggccctgctgctgcacgcagcaaggccacaggtgcagctgcagcagcctggcgcagagctggtgaagcctggcgccagcatgaagctgtcctgcaaggcctctggctacacattcacctcctattggatgcactgggtgaagcagcgcccaggccagggactggagtggatcggcatgatccaccccaactctgacaataccatctactatgagaagtttaagagcaaggccacactgaccgtggataagagctcctctacagcctacatgcagctgagctccctgacctccgaggactctgccgtgtactattgcgccatcatcatcacacccgtggtgcctaagttcgattattggggccagggcaccacactgaccgtgtctagcggaggaggaggaagcggaggaggagaatccggcggcggcggctctgacatcgtgatgacacagagccaccagtttatgagcacctccgtgggcgaccgggtgagcgtgacctgtaaggcctcccacgatgtgggcacctctgtggcctggtaccagcagaagccaggccagagccccaagctgctgatctattgggcctccacaaggcacaccggagtgccagaccgcttcacaggatctggaagcggcaccgacttcaccctgaccatcagcaacgtgcagtccgaggacctggccgattacttctgtcagcagtactcctcttatcctctgacatttggcgcaggaaccaagctggagctgaagagggcctctgatccaggctccggcggaggagaatcctgcccttacagcaacccatccctgtgctctggaggaggaggatcttgtccctatagcaatcctagcctgtgctccggcggaggaggcagcaccacaaccccagcaccaaggccacctacacctgcaccaaccatcgcatcccagccactgtctctgaggccagaggcatgcagacctgcagcaggcggcgccgtgcacaccagaggcctggacttcgcctgcgatatctacatctgggcacctctggcaggaacatgtggcgtgctgctgctgtccctggtcatcaccctgtattgtaagcgaggccggaagaaactgctgtatattttcaaacagccctttatgagacctgtgcagactacccaggaggaagacggctgcagctgtaggttccccgaggaagaggaaggcgggtgtgagctgagggtcaagtttagccgctccgcagatgcccctgcttaccagcaggggcagaatcagctgtataacgagctgaatctgggacggagagaggaatacgacgtgctggataaaaggcgcgggagagaccccgaaatgggaggcaagccacgacggaaaaacccccaggagggcctgtacaatgaactgcagaaggacaaaatggcagaggcctatagtgaaatcgggatgaagggagagagaaggcgcggcaaagggcacgatggcctgtaccaggggctgtctactgccaccaaggacacctatgatgctctgcatatgcaggcactgcctccaaggtgataatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaaatcggatcccccaggtagataagtagca

SEQ ID NO: 29 - DNA, 224 bp, artificial
  BGH poly A sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 30 - DNA, 301 bp, artificial
  B2M Left homology arm
cgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactc

SEQ ID NO: 31 - DNA, 300 bp, artificial
  B2M Right homology arm
tctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaag

SEQ ID NO: 32 - DNA, 2781 bp, artificial
  TRAC TALEN left Nucl. Seq
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 33 - DNA, 2781 bp, artificial
  TRAC TALEN right Nucl. Seq
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 34 - DNA, 2781 bp, artificial
  B2M TALEN left Nucl. Seq
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 35 - DNA, 2781 bp, artificial
  B2M TALEN right Nucl. Seq
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 36 - protein, 925 aa, artificial
  TRAC TALEN left Protein. Seq
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 37 - protein, 925 aa, artificial
  TRAC TALEN right Prot. Seq
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 38 - protein, 925 aa, artificial
  B2M TALEN left Prot. Seq
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 39 - protein, 925 aa, artificial
  B2M TALEN right Prot. Seq
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 40 - protein, 74 aa, artificial
  i53 Prot. Seq
MLIFVKTLTGKTITLEVEPSDTIENVKAKIQDKEGIPPDQQRLAFAGKSLEDGRTLSDYNILKDSKLHPLLRLR

SEQ ID NO: 41 - protein, 233 aa, artificial
  Bclxl Prot.Seq
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 42 - protein, 7 aa, artificial
  B2M Signal Seq
SLSGLEA

SEQ ID NO: 43 - protein, 9 aa, artificial
  HLA-G peptid
VMAPRTLIL

SEQ ID NO: 44 - protein, 15 aa, artificial
  3xG4S linker
GGGGSGGGGSGGGGS

SEQ ID NO: 45 - protein, 99 aa, artificial [sequence omitted]

SEQ ID NO: 46 - protein, 20 aa, artificial
  4xG4S linker
GGGGSGGGGSGGGGSGGGGS

SEQ ID NO: 47 - protein, 335 aa, artificial
  HLA-E  full length
HSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYYKAEWSDSAQGSESHSL

SEQ ID NO: 48 - DNA, 225 bp, artificial
  i53 Nucl. Seq
atgttgattttcgtgaaaacccttaccgggaaaaccatcaccctcgaggttgaaccctcggatacgatagaaaatgtaaaggccaagatccaggataaggaaggaattcctcctgatcagcagagactggcctttgctggcaaatcgctggaagatggacgtactttgtctgactacaatattctaaaggactctaaacttcatcctctgttgagacttcgttga

SEQ ID NO: 49 - DNA, 702 bp, artificial
  Bclxl Nucl.Seq
atgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

(19) United States
(12) Patent Application Publication
(10) Pub. No.: US 2023/0416786 A1
(43) Pub. Date: Dec. 28, 2023

(54) USE OF AMINOQUINOLINE COMPOUNDS FOR HIGHER GENE INTEGRATION

(71) Applicant: CELLECTIS SA, Paris (FR)

(72) Inventors: Alexandre JUILLERAT, New York, NY (US); Philippe DUCHATEAU, Paris (FR); Ming YANG, New York, NY (US)

(21) Appl. No.: 18/253,977

(22) PCT Filed: Nov. 30, 2021
(86) PCT No.: PCT/EP2021/083539

§ 371 Date: May 23, 2023

Related U.S. Application Data
(60) Provisional application No. 63/119,302, filed on Nov. 30, 2020.

(30) Foreign Application Priority Data
Feb. 25, 2021 (DK) PA202170087

(51) Int. Cl. C12N 15/90; C12N 9/22; C12N 15/86; C07D 215/46

(52) U.S. Cl. CPC C12N 15/907; C12N 9/22; C12N 15/86; C07D 215/46; C12N 2750/14143

(57) ABSTRACT

The invention provides aminoquinoline compounds as powerful enhancers of genetic recombination in living cells, especially to perform site-directed gene integration of exogenous DNA template by homologous recombination. In particular, disclosed are methods by which cells are treated with chloroquine and/or hydroxychloroquine prior to, or concomitantly with, the introduction of exogenous DNA templates, and optionally in presence of rare-cutting endonucleases, to obtain higher rates of gene integration or correction.

### FIELD OF THE INVENTION

The present invention pertains to the field of gene editing methods and gene therapy, in which efficiency of transgene integration and gene repair still needs to be improved.

The invention provides aminoquinoline compounds as powerful enhancers of genetic recombination in living cells, especially to perform site-directed gene integration of exogenous DNA template by homologous recombination. In particular, disclosed are methods by which cells are treated with chloroquine and/or hydroxychloroquine prior to, or concomitantly with, the introduction of exogenous DNA templates, and optionally in presence of rare-cutting endonucleases, to obtain higher rates of gene integration or correction.

### BACKGROUND OF THE INVENTION

Aminoquinoline compounds have been used for decades as primary and most successful drugs against malaria.

However, besides their antiparasitic properties, these molecules are known to display pleiotropic effects on cells, especially observed in the context of viral infections, which have not been all elucidated and remain controversial.

Chloroquine and hydroxychloroquine, the most studied aminoquinoline compounds, exert direct antiviral effects, inhibiting pH-dependent steps of the replication of several viruses including members of the flaviviruses, retroviruses, and coronaviruses. Their best-studied effects are those against HIV replication, which are being tested in clinical trials.

The mechanism of the anti-HIV effects of chloroquine/hydroxychloroquine is a reduction in the infectivity of newly produced virions [reviewed in Savarino et al. (2001) The anti-HIV-1 activity of chloroquine. J Clin Virol. 20:131-135]. The antiviral effects of chloroquine are associated with the reduced production of the heavily glycosylated epitope 2G12, which is located on the gp120 envelope glycoprotein surface and is fundamental for virus infectivity. These effects are likely to be attributed to the increased pH of lysosomal and trans-Golgi network, which impairs the function of glycosyl-transferases involved in the post-translational processing of the HIV glycoproteins. As viral envelope glycosylation is mediated by cellular enzymes, its inhibition may explain the broad spectrum of the in-vitro anti-HIV activity of chloroquine against all major subtypes of HIV-1 and HIV-2. The effect of chloroquine/hydroxychloroquine on cellular rather than viral enzymes may also result in a low propensity to resistance development. On another hand, chloroquine has immunomodulatory effects, suppressing the production/release of tumour necrosis factor α and interleukin 6, which may be useful to mediate the inflammatory complications of several viral diseases, such as in Severe acute respiratory syndrome (SARS) [Keyaerts, E. et al. (2004) In vitro inhibition of severe acute respiratory syndrome coronavirus by chloroquine Biochem Biophys Res Commun. 323(1): 264-268].

Some research groups have investigated the effects of chloroquine against different types of cancer cells, but so far with limited success. Several clinical studies have been initiated to test the effects of chloroquine as adjuvant in chemotherapy treatments to sensitise neoplastic cells to radiation, especially to target treatment-refractory glioblastoma cancers. Interest to chloroquine as an adjuvant treatment for glioblastoma was sparked by the initial observation that addition of chloroquine to standard therapy led to a significant prolongation of survival in patients with glioblastoma cancers, where chloroquine could potentiate cytotoxicity of temozolomide (TMZ) and ionizing radiation in glioma cells [De Ruysscher D. et al., (2018) The Addition of Chloroquine to Chemoradiation for Glioblastoma (CHLOROBRAIN) U.S. National Library of Medicine]. The mechanisms of radio- or chemo-sensitization mediated by chloroquine in glioma cells are however not entirely understood. Modulation of the autophagic response, by which the cell allows the orderly degradation and recycling of its unnecessary or dysfunctional cellular components, remains the most intensively investigated mechanism of chloroquine in non-neoplastic and cancer cells. It has been shown that Glioma resistant cells possess an augmented DNA damage response (DDR), which renders them capable of surviving cytotoxic treatments giving them the ability to escape from the cytotoxic effect of radiation. From these observations, it is believed that chloroquine has an intrinsic genome repair-inhibiting activity manifested in different types of normal and neoplastic cells in-vitro and in-vivo [Liu E. et al. (2015) Loss of autophagy causes a synthetic lethal deficiency in DNA repair (2015) PNAS 112:773-78]. Although the exact mechanisms of chloroquine-mediated inhibition of DNA repair remain unknown, they are likely to reflect the causative relationship between impaired autophagy and deficient DNA repair [Weyerhäuser P, et al. (2018) Re-purposing Chloroquine for Glioblastoma: Potential Merits and Confounding Variables. Front. Oncol. 8:335]. In the present invention, chloroquine and hydroxychloroquine have surprisingly proven to dramatically help gene integration into cells by way of their own gene repair mechanisms, especially by homologous recombination, which was unexpected. As shown in the experimental results obtained by the inventors, the effect of the aminoquinoline compounds on transgene integration was most significant in hematopoietic stem cells (HSC) as well as in other blood cells, making them useful in gene editing strategies for gene therapy.

### SUMMARY OF THE INVENTION

It is to be understood that both the foregoing general description of the embodiments and the following detailed description are exemplary, and thus do not restrict the scope of the embodiments.

The present invention lies, at least in part, in the use of aminoquinoline compound(s), as usually used in anti-malaria treatments, to increase the frequency of targeted genome modification in cells. To the inventor's knowledge this is the first time that such compounds are used in combination with sequence-specific genome editing reagents to perform gene editing in living cells. This invention is particularly useful in view of manufacturing engineered blood cells for gene therapy as shown in the experimental section herein, and more specifically to modify hematopoietic stem cells (HSCs). Nevertheless, it can be broadly applied for the genome engineering of most cell types.

The ability to manipulate any genomic sequence by gene editing has created diverse opportunities to treating many different diseases and disorders. Recent progress in genome editing technologies based on programmable nucleases such as transcription activator-like effector nucleases (TALEN), and clustered regularly interspaced short palindromic repeat (CRISPR)-associated nuclease Cas9 are opening the possibility of achieving therapeutic genome editing, resulting deletion of target genes (knock-out) or precise insertion of exogenous sequences (knock-in).

Whereas gene knock-out rates can usually reach over 90% in programmable nuclease treated cells, the efficiency of gene knock-in is lagging behind. Moreover, genetically modified cells are sometimes almost phenotypically indistinguishable from normal counterparts, making screening and isolating positive cells rather challenging and time-consuming. The present methods to improve gene knock-in efficiency, which can generate high purity knock-in cell populations of therapeutic grade, will certainly benefit the manufacturing of cell therapy products.

Here, the invention seeks to improve gene knock-in efficiency in primary human cells using small molecule treatments. We demonstrate that chloroquine, as well as its derivative hydroxychloroquine, and likely other small molecules in the same class, can significantly improve targeted gene knock-in.

In one aspect, the invention provides with methods to increase the frequency of targeted integration into the genome of a cell, characterized in that said methods comprise the step of treating the cell with a sequence-specific nuclease or nickase reagent and at least one aminoquinoline compound(s).

In particular, the invention provides with various methods for targeted integration at a selected locus into cells by using exogenous nucleic acid template(s), said methods comprising, for instance, one or several of the step of:
i) contacting the cells with aminoquinoline compound(s);
ii) introducing into said cells at least one sequence-specific nuclease or nickase reagent that specifically targets said selected locus,
iii) introducing into said cells a nucleic acid template to be integrated at said locus,
iv) cultivating the cells to induce DNA repair and integration of the nucleic acid template at said selected locus targeted by said sequence-specific nuclease or nickase;
v) optionally, selecting the cells which have integrated the nucleic acid template.

Beside producing gene edited therapeutic cells or creating engineered cell lines, the invention contemplates treating the cells with an aminoquinoline compound to improve genome scale engineering and analysis, such as in the case of oligonucleotide capture assays (OCA), which measures the level of integration of labelled oligonucleotide probes into the genome when using gene editing reagents in cells (detection of off-target sites).

In another aspect, the invention is directed to cell culture media, electroporation media or buffers, therapeutic compositions, kits, or nanoparticles, to be used to perform the invention comprising at least an aminoquinoline compound as defined herein. Such compositions can optionally comprise a nucleic acid template(s) to be integrated into the genome of the cell(s) and/or a sequence specific gene editing reagent, preferably a rare-cutting endonuclease.

Given the fact that Aminoquinoline compounds, such as chloroquine and hydroxychloroquine, have a well-studied toxicity profile and that the half-century-long use of this drug in the therapy of malaria has demonstrated the safety of acute administration of chloroquine to human beings, even of a high dosage of the drug (up to 500 mg of chloroquine base per day) and during pregnancy [Klinger G, et al. (2001) Ocular toxicity and antenatal exposure to chloroquine or hydroxychloroquine for rheumatic diseases. Lancet. 358:813-814], makes the methods of the invention safe for the production of therapeutic grade cells and further for their use in gene therapy.

### BRIEF DESCRIPTION OF THE FIGURES

FIG. 1: Schematic representation of examples of nuclease-induced targeted integration strategies that are applied in HSCs and/or T cells by treating the cells with aminoquinoline compounds as per the present invention. A: TALEN are used as gene editing reagent to cleave the B2M locus for the targeted integration of an HLA-E construct. This Integration leads to the inactivation of endogenous B2M expression, whereas expression of this HLA-E construct allows T-cells to escape destruction by NK cells. B: TALEN are used as gene editing reagent to cleave the TRAC locus for the targeted integration of a polynucleotide encoding an anti-mesothelin chimeric antigen receptor (MESO-CAR construct) allowing TCR inactivation (to make allogeneic T-cells less alloreactive) and CAR expression. This results into allogeneic CAR-T cells that target mesothelin positive malignant cells.

FIG. 2: Experimental results showing that chloroquine as used according to the present invention stimulates targeted integration of HLA-E construct at the B2M locus in HSCs. Flow cytometry analysis of HSC treated in different conditions detailed in Example 2. A: untreated, B: B2M TALEN treated, C: B2M TALEN+AAV treated (comprising HLA-E construct) D: B2M TALEN+AAV treated in presence of chloroquine (+CQ). Lower panel E: graphic comparing the percentages of HLA-E positive HSCs cells obtained with the different conditions tested in A, B, C and D.

FIG. 3: Experimental results showing that chloroquine stimulates targeted integration of CAR construct at the TRAC locus in primary T-cell as detailed in Example 3. Percentage of CAR positive T-cells obtained after TRAC TALEN+DNA repair template treatment at the different chloroquine concentrations tested. UT: untreated cells.

FIG. 4: Experimental results showing that chloroquine stimulates nuclease induced targeted integration at different concentration tested. Percentage of HLA-E positive HSCs obtained after treatment of B2M TALEN+HLA-E AAV in presence of 0, 0.01, 0.02, 0.04 or 0.1 nM of chloroquine.

FIG. 5: Flow cytometry analysis of HSCs treated with chloroquine (CQ) and hydroxychloroquine (HCQ) as detailed in Example 5. The results show that both CQ and HCQ stimulate nuclease induced targeted integration. A: untreated HCSs; B: HSCs treated with B2M TALEN and HLA-E AAV (without aminoquinoline compounds); C: treatment of HSC with CQ, B2M TALEN and HLA-E AAV; D: treatment of HSC with HCQ, B2M TALEN and HLA-E AAV.

FIG. 6: Flow cytometry analysis of HSCs treated with chloroquine (CQ) and B2M TALEN as gene editing reagent as detailed in Example 6. B2M TALEN with or without mRNAs encoding i53 are co-electroporated before transduction with HLA-E AAV. The results show that chloroquine can potentiate know gene repair stimulators factors, referred to herein as gene repair reagents. A: untreated HCSs; B: HSCs treated with: B2M TALEN and HLA-E AAV (without aminoquinoline compounds); C: HSCs treated with: B2M TALEN, HLA-E AAV with CQ; D: HSCs treated with: B2M TALEN and i53 mRNAs and HLA-E AAV (without aminoquinoline compounds); E: HSCs treated with: B2M TALEN and i53 mRNAs and HLA-E AAV with CQ.

FIG. 7: Schematic representation of relevant genes that can be targeted by the methods of the present invention to promote targeted gene integration in order to address inherited pathologies or cancer by obtaining gene integration, correction or replacement in the genome of HSCs or in their differentiated cell types. The pathologies related to the genes are detailed below. These diseases have been treated so far by bone marrow transfer from healthy donors to compatible patients [Morgan R. A., et al. (2017) Hematopoietic Stem Cell Gene Therapy: Progress and Lessons Learned. Cell Stem Cell. 21(5):574-590]:
HSCs: Fanconi Anemia (FANC A-F).
Platelets: Hemophilia A (Factor VIII (F8)); Hemophilia B (Factor IX (F9)); Factor X deficiency (Factor X (F10)); Wiskott-Aldrich Syndrome (Wiskott Aldrich Syndrome Protein (WASP)).
Neutrophils: X-linked Chronic Granulomatous Disease (Cytochrome B-245 Beta Chain (CYBB)); Kostmann's Syndrome (Elastase Neutrophil Expressed (ELANE)).
Erythrocytes: Alpha-Thalassemia (Hemoglobin Subunit Alpha (HBA)); Beta-Thalassemia and Sickle Cell Disease (Hemoglobin Subunit Beta (HBB)); Pyruvate Kinase Deficiency (Pyruvate Kinase, Liver and RBC (PKLR)); Diamond-Blackfan Anemia (Ribosomal Protein S19 (RPS19)).
Monocytes: X-linked Adrenoleukodystrophy (ATP Binding Cassette Subfamily D Member 1 (ABCD1)); Metachromatic Leukodystrophy (Arylsulfatase A (ARSA)); Gaucher disease (Glucosylceramidase Beta (GBA)); Hunter Syndrome (Iduronate 2-Sulfatase (IDS)); Mucopolysaccharidosis type I (Iduronidase, Alpha-L (IDUA)); Osteopetrosis (T-Cell Immune Regulator 1 (TCIRG1)).
B Cells: Adenosine deaminase (ADA)-deficient Severe Combined Immunodeficiency (Adenosine Deaminase (ADA)); X-linked severe combined immunodeficiency (Interleukin 2 Receptor Subunit Gamma (IL2RG)); Wiskott-Aldrich Syndrome (Wiskott RECTIFIED SHEET (RULE 91) ISA/EP Aldrich Syndrome Protein (WASP)); X-linked agammaglobulinemia (Bruton's Tyrosine Kinase (BTK)).
T Cells: Adenosine Deaminase (ADA)-deficient Severe Combined Immunodeficiency (ADA); X-linked severe combined immunodeficiency (IL2RG); Wiskott-Aldrich Syndrome Protein (WASP); X-linked Hyper IgM syndrome (CD40 Ligand (CD40LG)); IPEX Syndrome (Forkhead Box P3 (FOXP3)); Early Onset Inflammatory \Disease (Interleukin 4, 10, 13 (IL-4, 10, 13)); Hemophagocytic Lymphohistiocytosis (Perforin 1 (PRF1)); Cancer and infection (T-cell receptor (TCR); chimeric antigen receptors (CAR)).

FIG. 8: Schematic representation of the different DNA repair pathways used by the cells to repair DNA breaks upon double strand break induced by a gene editing reagent. According to the invention, key proteins can be over expressed in the cells upon treatment with an aminoquinoline compound to stimulate gene insertion/correction through the different pathways, in particular to promote homologous recombination (HR). Combining an aminoquinoline compound with a gene repair reagent, such as one of the key proteins referred to in this table, to improve gene insertion or correction is an aspect of the present invention.

### DETAILED DESCRIPTION OF THE INVENTION

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2nd edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Unless specifically defined herein, all technical and scientific terms used have the same meaning as commonly understood by a skilled artisan in the fields of gene therapy, biochemistry, genetics, immunology, cancer and molecular biology. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means plus or minus 10% of the numerical value of the number with which it is being used.

The present invention is drawn to the use of aminoquinoline compound(s) to increase the frequency of targeted genome modification in a cell.

By “targeted genome modification” is meant non-randomly introducing a mutation at a specific locus, which may have various incidence on the genome, such as inactivating a genomic sequence, inserting an exogenous nucleic acid sequence, replacing at least one nucleotide to obtain gene correction. In general, the targeted modification is performed at a selected locus (loci), more generally at a locus which is predetermined and/or specified by a sequence-specific “gene editing reagent”.

By “gene editing reagent” is meant a molecular entity that participates to an enzyme reaction acting on a polynucleotide molecular structure alone or by forming a complex with another molecular entity, in such a way that a mutation can be induced. Examples of such gene editing reagents are a component of a CRISPR complex, RNA guide or RNA guided endonuclease, and molecular entities allowing the activity on genomic DNA of rare-cutting endonucleases, reverse transcriptases, fusion nickases and base editors (deaminase) such as reviewed for instance by Sakata, R. C. et al. [Base editors for simultaneous introduction of C-to-T and A-to-G mutations (2020) Nat. Biotechnol. 38, 865-869].

In certain aspects of the invention the cells are treated with an aminoquinoline compound(s) to increase the targeted integration at a locus of an exogenous nucleic acid template.

By exogenous nucleic acid template (“donor template”) is meant an artificial polynucleotide sequence that has been designed to be incorporated into the genome at the locus. The nucleic acid template may not be fully integrated into the genome but only partially depending on the cell mechanisms relied upon to obtain recombination of the template with the endogenous locus sequence (ex: Homologous recombination, NHEJ, . . . ) and the gene editing reagents selected by the operator.

The donor templates according to the present invention are generally polynucleotide sequences which can be included into a variety of vectors described in the art prompt to deliver the donor templates into the nucleus at the time the endonuclease reagents get active to obtain their site directed insertion into the genome generally by NHEJ or homologous recombination.

In preferred embodiments, said exogenous nucleic acid template to be integrated at said locus is comprised into a non-integrative viral vector such as an IDLV or AAV [Naso M. F., et al. (2017) Adeno-Associated Virus (AAV) as a Vector for Gene Therapy. BioDrugs. 31(4):317-334], more especially from the AAV6 family as described for instance in WO2018073391.

Still according to this broad aspect, the invention more particularly provides a method of insertion of an exogenous nucleic acid sequence into an endogenous polynucleotide sequence in a cell, comprising at least the steps of:
transducing into said cell an AAV vector comprising said exogenous nucleic acid sequence and sequences homologous to the targeted endogenous DNA sequence, and
introducing a sequence specific endonuclease or nickase reagent to cleave said endogenous sequence at the locus of insertion.

The obtained insertion of the exogenous nucleic acid sequence may result into the introduction of genetic material, correction or replacement of the endogenous sequence, more preferably “in frame” with respect to the endogenous gene sequences at that locus.

According to another aspect of the invention, from 10³ to 10⁷ preferably from 10⁴ to 10⁵, more preferably about 10⁴ viral genomes are transduced per cell.

As one object of the present invention, the AAV vector used in the method can comprise a promoterless exogenous coding sequence as any of those referred to in this specification in order to be placed under control of an endogenous promoter at one loci selected among those listed in the present specification.

As an object of the present invention, the AAV vector used in the method can comprise a 2A peptide cleavage site followed by the cDNA (minus the start codon) forming the exogenous coding sequence.

By “exogenous” is meant that the sequence or mutation that is to be integrated into the cell genome was not originally present into the cell genome at this locus. This does not mean that the sequence can not be found elsewhere in the genome of the treated cell.

In preferred embodiments, the aminoquinoline compound is used in combination with a gene editing reagent that has endonuclease or nickase activity, which is preferably a sequence-specific gene editing reagent, and more preferably a rare-cutting endonuclease inducing a double-strand break at a specific locus such as a such a RNA-guided endonuclease, TALE-nuclease, mega-TALE, Zing-finger nuclease (ZFN) or engineered homing endonucleases, as described below. In preferred embodiments, the gene editing reagent has a nickase activity on one or two nucleotide strands, such as preferentially Cas9 paired nickases as described in WO2014191518 or fusion nickases as described for instance by Rees H. A. et al. [Development of hRad51-Cas9 nickase fusions that mediate HDR without double-stranded breaks. Nat. Commun. (2019) 10: 2212].

Endonucleases can be classified as rare-cutting endonucleases when having typically a polynucleotide recognition site greater than 10 base pairs (bp) in length. In some embodiments the rare-cutting endonuclease has a recognition site of from 14-55 bp. Rare-cutting endonucleases significantly increase homologous recombination by inducing DNA double-strand breaks (DSBs) at a defined locus thereby allowing gene repair or gene insertion therapies [Pingoud, A. and G. H. Silva (2007). Nat. Biotechnol. 25(7): 743-4)]

The nuclease reagents of the invention are generally “sequence-specific reagents”, meaning that they can induce DNA cleavage in the cells at predetermined loci, referred to by extension as “targeted gene”. The nucleic acid sequence which is recognized by the sequence specific gene editing reagents is referred to as “target sequence”. Said target sequence is usually selected to be rare or unique in the cell's genome, and more extensively in the human genome, as can be determined using software and data available from human genome databases, such as http://www.ensembl.org/index.html.

“Rare-cutting endonucleases” are sequence-specific gene editing reagents of choice, herewith also covered by the terms “sequence-specific endonuclease reagent”, insofar as their recognition sequences generally range from 10 to 50 successive base pairs, preferably from 12 to 30 bp, and more preferably from 14 to 20 bp.

According to a preferred aspect of the invention, said endonuclease reagent is a nucleic acid encoding an “engineered” or “programmable” rare-cutting endonuclease, such as a homing endonuclease as described for instance by Arnould S., et al. (WO2004067736), a zing finger nuclease (ZFN) as described, for instance, by Urnov F., et al. (Highly efficient endogenous human gene correction using designed zinc-finger nucleases (2005) Nature 435:646-651), a TALE-Nuclease as described, for instance, by Mussolino et al. (A novel TALE nuclease scaffold enables high genome editing activity in combination with low toxicity (2011) Nucl. Acids Res. 39(21):9283-9293), or a MegaTAL nuclease as described, for instance by Boissel et al. (MegaTALs: a rare-cleaving nuclease architecture for therapeutic genome engineering (2013) Nucleic Acids Research 42 (4):2591-2601).

According to another embodiment, the endonuclease reagent is a RNA-guide to be used in conjunction with a RNA guided endonuclease, such as Cas9 or Cpf1, as per, inter alia, the teaching by Doudna, J., and Chapentier, E., (The new frontier of genome engineering with CRISPR-Cas9 (2014) Science 346 (6213):1077), which is incorporated herein by reference.

According to a preferred aspect of the invention, the endonuclease reagent is transiently expressed into the cells, meaning that said reagent is not supposed to integrate into the genome or persist over a long period of time, such as be the case of RNA, more particularly mRNA, proteins or complexes mixing proteins and nucleic acids (eg: Ribonucleoproteins).

An endonuclease under mRNA form is preferably synthetized with a cap to enhance its stability according to techniques well known in the art, as described, for instance, by Kore A. L., et al. (Locked nucleic acid (LNA)-modified dinucleotide mRNA cap analogue: synthesis, enzymatic incorporation, and utilization (2009) J Am Chem Soc. 131(18):6364-5).

Due to their higher specificity, TALE-nucleases have proven to be particularly appropriate sequence specific nuclease reagents for therapeutic applications, especially under heterodimeric forms—i.e. working by pairs with a “right” monomer (also referred to as “5′” or “forward”) and ‘left” monomer (also referred to as “3″” or “reverse”) as reported for instance by Mussolino et al. (TALEN® facilitate targeted genome editing in human cells with high specificity and low cytotoxicity (2014) Nucl. Acids Res. 42(10): 6762-6773).

As previously stated, the sequence specific reagent is preferably under the form of nucleic acids, such as under DNA or RNA form encoding a rare cutting endonuclease a subunit thereof, but they can also be part of conjugates involving polynucleotide(s) and polypeptide(s) such as so-called “ribonucleoproteins”. Such conjugates can be formed with reagents as Cas9 or Cpf1 (RNA-guided endonucleases) as recently respectively described by Zetsche, B. et al. (Cpf1 Is a Single RNA-Guided Endonuclease of a Class 2 CRISPR-Cas System (2015) Cell 163(3): 759-771).

“Exogenous sequence” refers to any nucleotide or nucleic acid sequence that was not initially present at the selected locus in the genome of the cell to be treated. This sequence may be homologous to, or a copy of, a genomic sequence, or be a foreign sequence introduced into the cell. By opposition “endogenous sequence” means a cell genomic sequence initially present at a locus. The exogenous sequence preferably codes for a polypeptide which expression confers a therapeutic advantage over sister cells that have not integrated this exogenous sequence at the locus. An endogenous sequence that is gene edited by the insertion of a nucleotide or polynucleotide as per the method of the present invention, in order to express a different polypeptide is broadly referred to as an exogenous coding sequence

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

By “mutation” is intended the substitution, deletion, insertion of up to one, two, three, four, five, six, seven, eight, nine, ten, eleven, twelve, thirteen, fourteen, fifteen, twenty, twenty five, thirty, forty, fifty, or more nucleotides/amino acids in a polynucleotide (cDNA, gene) or a polypeptide sequence. In some embodiments, the mutation can affect the coding sequence of a gene or its regulatory sequence. It may also affect the structure of the genomic sequence or the structure/stability of the encoded mRNA.

As used herein, the term “locus” is the specific physical location of a DNA sequence (e.g. of a gene) into a genome. The term “locus” can refer to the specific physical location of a rare-cutting endonuclease target sequence on a chromosome or on an infection agent's genome sequence. Such a locus can comprise a target sequence that is recognized and/or cleaved by a sequence-specific endonuclease according to the invention. It is understood that the locus of interest, in the present invention, can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.

The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA RNA hybrid cleavage can result in the production of either blunt ends or staggered ends.

“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

As used herein, the term “aminoquinoline compounds” refers to aminoquinoline derivatives, such as those known and described to exert anti-malaria activity in the literature, more particularly those derivatives of 4-aminoquinoline and 8-aminoquinoline. The principal biological activity of 8-aminoquinolines is thought to be due to highly reactive metabolites such as the 5-methoxy metabolite. Preferred representatives of 8-aminoquinolines for the purpose of the invention are pamaquine, primaquine, bulaquine and tafenoquine [Recht, I. et al. (2014) Safety of 8-aminoquinoline antimalarial medicines. World Health Organization. V. Mahidol Oxford Research Unit. ISBN 978 92 4 150697 7]. Preferred representatives of 4-aminoquinolines for the purpose of the present invention are those of formula 1, with R groups ranging from simple H or Cl atoms to alkyl substitutions and trifluoromethyl groups, and n either 0 or 1, such as amodiaquine, chloroquine, and hydroxychloroquine.

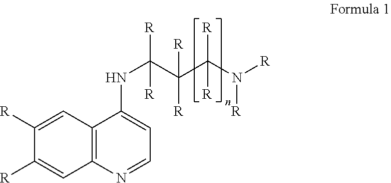

As used herein, the term “chloroquine” or “choloroquine compounds” includes chloroquine-like compounds, chloroquine and enantiomers, analogs, derivatives, metabolites, pharmaceutically acceptable salts, and mixtures thereof. Examples of chloroquine compounds include, but are not limited to, chloroquine phosphate, hydroxychloroquine, chloroquine diphosphate, chloroquine sulphate, hydroxychloroquine sulphate, and enantiomers, analogs, derivatives, metabolites, pharmaceutically acceptable salts, and mixtures thereof. The term “chloroquine-like compounds” as used herein means compounds that mimic chloroquine's biological and/or chemical properties.

Examples of suitable chloroquine compounds include chloroquine phosphate; 7-chloro-4-(4-diethylamino-1-butylamino)quinoline (desmethylchloroquine); 7-hydroxy-4-(4-diethylamino-1-butylamino)quinoline; 7-chloro-4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-hydroxy-4-(1-carboxy-4-diethylamino-1-methylbutylamino)quinoline; 7-chloro-4-(4-ethyl-(2-hydroxyethyl)-amino-1-methylbutylamino)quinoline (hydroxychloroquine); 7-hydroxy-4-(4-ethyl-(2-hydroxyethyl)-amino-1-methyl-1-butylamino)quinoline; hydroxychloroquine phosphate; 7-chloro-4-(4-ethyl-(2-hydroxyethyl)-amino-1-butylamino)quinoline (desmethylhydroxychloroquine); 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-hydroxy-4-(1-carboxy-4-ethyl-(-2-hydroxyethyl)-amino-1-methylbutylamino)quinoline; 8-[(4-aminopentyl)amino]-6-methoxydihydrochloride quinoline; 1-acetyl-1,2,3,4-tetrahydroquinoline; 8-[4-aminopentyl)amino]-6-methoxyquinoline dihydrochloride; 1-butyryl-1,2,3,4-tetrahydroquinoline; 7-chloro-2-(o-chlorostyryl)-4-[4-diethylamino-1-methylbutyl]aminoquinoline phosphate; 3-chloro-4-(4-hydroxy-.alpha.,.alpha.′-bis(2-methyl-1-pyrrolidinyl)-2,5-xylidinoquinoline, 4-[(4-diethylamino)-1-methylbutyl)amino]-6-methoxyquinoline; 3,4-dihydro-1(2H)-quinolinecarboxyaldehyde; 1,1′-pentamethylenediquinoleinium diiodide; and 8-quinolinol sulfate, enantiomers thereof, as well as suitable pharmaceutical salts thereof.

As mentioned above, the chloroquine compounds useful herein include chloroquine analogs and derivatives. A number of chloroquine analogs and derivatives are well known. For example, suitable compounds and methods for synthesizing the same are described in U.S. Pat. Nos. 6,417,177; 6,127,111; 5,639,737; 5,624,938; 5,736,557; 5,596,002; 5,948,791; 2,653,940; 2,233,970; 5,668,149; 5,639,761; 4,431,807; and 4,421,920.

Additional suitable chloroquine derivatives include aminoquinoline derivatives and their pharmaceutically acceptable salts such as those described in U.S. Pat. Nos. 5,948,791 and 5,596,002. Suitable examples include (S)—N₂-(7-Chloro-quinolin-4-yl)-N₁,N₁-dimethyl-propane-1,2-diamine; (R)—N₂-(7-chloro-quinolin-4-yl)-N₁,N₁-dimethyl-propane-1,2-diamine; N₁-(7-chloro-quinolin-4-yl)-2,N₂,N₂-trimethyl-propane-1,2-diamine; N₃-(7-chloro-quinolin-4-yl)-N₁,N₁-diethyl-propane-1,3-diamine; (RS)-(7-chloro-quinolin-4-yl)-(1-methyl-piperidin-3-yl)-amine; (RS)-(7-chloro-quinolin-4-yl)-(1-methyl-pyrrolidin-3-yl)-amine; (RS)—N₂-(7-Chloro-quinolin-4-yl)-N₁,N₁-dimethyl-propane-1,2-diamine; (RS)—N₂-(7-chloro-quinolin-4-yl)-N₁,N₁-diethyl-propane-1,2-diamine; (S)—N₂-(7-chloro-quinolin-4-yl)-N₁,N₁-diethyl-propane-1,2-diamine; (R)—N₂-(7-chloro-quinolin-4-yl)-N₁,N₁-diethyl-propane-1,2-diamine; (RS)-7-chloro-quinolin-4-yl)-(1-methyl-2-pyrrolidin-1-yl-ethyl)-amine; N₂-(7-chloro-quinolin-4-yl)-N₁,N₁-dimethyl-ethane-1,2-diamine; N₂-(7-chloro-quinolin-4-yl)-N₁,N₁-diethyl-ethane-1,2-diamine; N₃-(7-chloro-quinolin-4-yl)-N₁,N₁-dimethyl-propane-1,3-diamine; (R)—N-(7-chloro-quinolin-4-yl)-N₂,N₂-dimethyl-propane-1,2-diamine; (S)—N-(7-chloro-quinoline-4-yl)-N₂,N₂-dimethyl-propane-1,2-diamine; (RS)-(7-chloro-quinolin-4-yl)-(1-methyl-pyrrolidin-2-yl-methyl)-amine; N₁-(7-Chloro-quinolin-4-yl)-N₂-(3-chloro-benzyl)-2-methyl-propane-1,2-diamine; N₁-(7-chloro-quinolin-4-yl)-N₂-(benzyl)-2-methyl-propane-1,2-diamine; N₁-(7-chloro-quinolin-4-yl)-N₂-(2-hydroxy-3-methoxy-benzyl)-2-methyl-propane-1,2-diamine; N₁-(7-chloro-quinolin-4-yl)-N₂-(2-hydroxy-5-methoxy-benzyl)-2-methyl-propane-1,2-diamine; and N₁-(7-chloro-quinolin-4-yl)-N₂-(4-hydroxy-3-methoxy-benzyl)-2-methyl-propane-1,2-diamine; (1S,2S)—N-(7-chloro-quinolin-4-yl)-N₂-(benzyl)-cyclohexane-1,2-diamine; (1S,2S)—N₁-(7-chloro-quinolin-4-yl)-N₂-(4-chlorobenzyl)-cyclohexane-1,2-diamine; (1S,2S)—N-(7-chloro-quinolin-4-yl)-N₂-(4-di methylamino-benzyl)-cyclohexane-1,2-diamine; cis-N₁-(7-chloro-quinolin-4-yl)-N₄-(4-dimethylamino-benzyl)-cyclohexane-1,4-diamine; cis-N₁-(7-chloro-quinolin-4-yl)-N₄-(benzyl)-cyclohexane-1,4-diamine; cis-N₁-(7-chloro-quinolin-4-yl)-N₄-(3-chloro-benzyl)-cyclohexane-1,4-diamine; cis-N-(7-chloro-quinolin-4-yl)-N₄-(2-hydroxy-4-methoxy-benzyl)-cyclohexane-1,4-diamine; cis-N₁-(7-chloro-quinolin-4-yl)-N₄-(3,5-dimethoxy-benzyl)-cyclohexane-1,4-diamine; cis-N₁-(7-chloro-quinolin-4-yl)-N₄-(4-methylsulphanyl-benzyl)-cyclohexane-1,4-diamine; cis-N₁-(7-chloro-quinolin-4-yl)-N₄-(4-diethylamino-benzyl)-cyclohexane-1,4-diamine; cis-N₁-(7-chloro-quinolin-4-yl)-N₄-(biphenyl-4-yl)methyl-cyclohexane-1,4-diamine; trans-N₁-(7-chloro-quinolin-4-yl)-N₄-[2-(3,5-dimethoxy-phenyl)-ethyl]-cyclohexane-1,4-diamine; cis-N₁-(7-chloro-quinolin-4-yl)-N₄-(4-methoxy-benzyl)-cyclohexane-1,4-diamine; trans-N₁-(7-chloro-quinolin-4-yl)-N₄-(4-dimethylamino-benzyl)-cyclohexane-1,4-diamine; and trans-N₁-(7-chloro-quinolin-4-yl)-N₄-(2,6-difluoro-benzyl)-cyclohexane-1,4-diamine.

Preferred examples of chloroquine compounds to be used as per the present invention are chloroquine diphosphate salt (N⁴-(7-Chloro-4-quinolinyl)-N¹,N¹-dimethyl-1,4-pentanediamine diphosphate salt, N4-(7-chloroquinolin-4-yl)-N1,N1-diethylpentane-1,4-diamine diphosphate) such as provided by Sigma under reference C6628, and hydroxychloroquine sulfate such as 7-Chloro-4-[4-(N-ethyl-N-b-hydroxyethylamino)-1-methylbutylamino]quinoline sulfate provided by Sigma under reference H9015.

Chloroquine and hydroxychloroquine are generally racemic mixtures of (−)- and (+)-enantiomers. The (−)-enantiomers are also known as (R)-enantiomers (physical rotation) and 1-enantiomers (optical rotation). The (+)-enantiomers are also known as (S)-enantiomers (physical rotation) and r-enantiomers (optical rotation). The metabolism of the (+)- and the (−)-enantiomers of chloroquine are described for instance in Augustijins and Verbeke [Clin. Pharmacokin. (1993) 24(3):259-69]. Preferably, the (−)-enantiomer of chloroquine is used. The enantiomers of chloroquine and hydroxychloroquine can be prepared by procedures known to the art.

In one aspect, the invention pertains to methods for targeted integration of an exogenous nucleic acid template at a selected locus into cells, said method comprising at least one step of:
i) contacting the cells with aminoquinoline compound(s);
ii) introducing into said cells at least one gene editing reagent that specifically targets said selected locus,
iii) introducing into said cells a nucleic acid template to be integrated at said locus,
iv) cultivating the cells to induce DNA repair and integration of the nucleic acid template at said selected locus targeted by said gene editing reagent;
v) optionally, selecting the cells which have integrated the nucleic acid template.

The above step can be performed in different orders depending on the involved gene editing methods. In some methods, the cells can be treated with the aminoquinoline compound after having introduced the exogenous nucleic acid template, preferably at the same time or before the gene editing reagent is introduced or expressed in the cell. In general, the aminoquinoline compound is added to the culture medium. Alternatively, the cells are transferred into a fresh medium comprising the aminoquinoline compound after an electroporation step introducing the gene editing reagent and/or the exogenous nucleic acid template.

Electroporation steps that are used to transfect immune cells are typically performed in closed chambers comprising parallel plate electrodes producing a pulse electric field between said parallel plate electrodes greater than 100 volts/cm and less than 5,000 volts/cm, substantially uniform throughout the treatment volume such as described in WO/2004/083379, which is incorporated by reference, especially from page 23, line 25 to page 29, line 11. One such electroporation chamber preferably has a geometric factor (cm⁻¹) defined by the quotient of the electrode gap squared (cm2) divided by the chamber volume (cm³), wherein the geometric factor is less than or equal to 0.1 cm⁻¹, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

In some embodiments, the gene editing method is carried out with two transfection steps, a first one to introduce the gene editing reagent and a second one to introduce the exogenous nucleic acid template, for instance, at least 5 to 15 hours later, preferably at least 10 to 15 hours later when a rare cutting endonuclease, is used as a reagent, such as a TALE-nuclease. The first and second steps can be performed for instance by electroporation. In some embodiments, the first electroporation consists in introducing mRNAs encoding a rare-cutting nickase or endonuclease as a gene editing reagent, and the second electroporation consists in introducing the nucleic acid template, such as a double stranded DNA. In some other embodiment, the first and second steps can be performed by electroporation and non-integrative viral transduction, electroporation consisting in introducing mRNAs encoding a rare-cutting nickase or endonuclease, and transduction consisting in introducing the exogenous nucleic acid template under the form of a viral vector, such as a AAV or IDLY. In such cases, it can be beneficial to have the aminoquinoline compound introduced between the two transfection steps, in the culture medium post electroporation and/or in the medium used for the transduction step also referred to as “transduction medium”.

The above method of the invention can also be performed in one step, in which the gene editing reagent and the exogenous nucleic acid template are concomitantly introduced in the cell (or allowing steps ii) and iii) to be performed at about the same time). For instance, both gene editing reagent and exogenous nucleic acid template can be introduced in the cell during the same delivery step (such as electroporation or transduction step) as described for instance by Sather et al. [Efficient modification of CCR5 in primary human hematopoietic cells using a megaTAL nuclease and AAV donor template (2015) Science Translational Medicine 7(307):307]. Alternatively, the electroporation of the gene editing reagent or polynucleotide encoding thereof can be performed shortly before or after, the non-integrative viral vector transduction. In another alternative both the gene editing reagent and the exogenous nucleic acid template may be transfected by using nanoparticles, such as silica based mesoporous particles as described for instance in WO2016124765. In still another alternative, a non-integrative viral vector can encode the gene editing reagent and also serve as an exogenous nucleic acid template. In such embodiments, the aminoquinoline compound can be directly introduced in the nanoparticles or in any transition culture medium used during or after transfection/transduction steps.

As referred to before, the gene editing reagent in the methods of the present invention is preferably a sequence-specific nickase or endonuclease, which is usually expressed in the cell upon introduction by electroporation of a polynucleotide encoding thereof. In this respect mRNA are preferentially used for obtaining transient expression of the gene editing reagents.

In some aspects of the invention, the nucleic acid template is DNA polynucleotide provided as a plasmid. In some other aspects, said nucleic acid template can be double stranded (dsDNA), such as a PCR product, with a length preferably of more than 2 kb, preferably more than 2.5 kb, more preferably more than 3 kb, even more preferably between 2 and 10 kb. In further aspects, the nucleic acid template can be a single stranded polynucleotide, such as a short single-stranded oligodeoxynucleotide (ssODN).

In some aspects, the nucleic acid template to be integrated in the genome according to the present invention comprises the partial or complete nucleic acid sequence of a transgene to be expressed in the cell. By “transgene” is meant an exogenous gene sequence, generally a coding sequence, or a corrected or mutated version of an endogenous gene sequence.

From a therapeutic perspective, the exogenous nucleic acid template can comprise various gene sequences encoding therapeutic proteins, beneficial to patients in various indications. In preferred examples, the methods of the invention can be used to prepare engineered immune cells by integrating sequences encoding artificial ligands, receptors or antibodies, such as chimeric antigen receptor (CAR) or recombinant T-cells receptors (modified TCR).

In preferred embodiments of the present invention, the exogenous nucleic acid template is more than 200 bp, preferably more than 500 pb, and the integration of the nucleic acid template at said selected locus is obtained by homologous recombination. Without being bound by any theory, the inventors have hypothesized that aminoquinoline compounds take effect on enzymes involved in genome repair that would favour homologous recombination repair process(es), which could explain the higher rates of integration observed between treated and untreated cells during their experiments. Following this theory, the invention can be performed in any types of cells since the DNA repair pathways are almost universal in eucaryotic cells [Mladenov E. & LLiakis G. (2011) DNA repair: on the pathways to fixing DNA damage and errors—Edited by Francesca Storici—Intech Publishers DOI: 10.5772/24572]. Accordingly, the method of the cells in the present invention can be a plant or animal cell, preferably a primate cell, more preferably a human cell.

In some preferred embodiments, the invention allows the production of genetically engineered primary cells. Populations of primary cells are usually more difficult to transform than cell lines because they are more refractory to introduction of foreign macromolecules and have limited life span. Preferably, such cells, from patients or donors, are prepared ex-vivo before being administered to the patients.

By “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells. Primary cells are generally used in cell therapy as they are deemed more functional and less tumorigenic.

In general, primary immune cells are provided from donors or patients through a variety of methods known in the art, as for instance by leukapheresis techniques as reviewed by Schwartz J. et al. [Guidelines on the use of therapeutic apheresis in clinical practice-evidence-based approach from the Writing Committee of the American Society for Apheresis: the sixth special issue (2013) J Clin Apher. 28(3):145-284].

The primary immune cells according to the present invention can also be stem cells that have undergone differentiation, such as cord blood stem cells, progenitor cells, bone marrow stem cells, hematopoietic stem cells (HSC). Induced pluripotent stem cells (iPS) are also considered herein as primary cells for the purpose of the present invention.

The inventors have found that the present method was particularly suited to engineer blood cells, which are reputed refractory to gene integration, especially when using exogenous nucleic acid templates. The invention is thus particularly useful to engineer immune therapeutic cells, preferably lymphocytes obtainable from patients, such as preferably, macrophages, dendritic cells, T-cells or NK-cells.

According to a preferred embodiment, the present invention comprises methods to culture and transform hematopoietic stem cell (HSC). Treating or culturing hematopoietic stem cell (HSC) with aminoquinoline compounds has dramatically increased the success of gene integration in this type of cells, where usually the percentage of positive clones was remaining low. As shown in the experimental section, the rate of targeted gene integration obtained with the present invention reached a percentage above 35% and up to about 60% of positive cells in HSC populations. The present invention therefore aims to achieve more than 30%, preferably more than 35%, more preferably more than 40%, even more preferably 45% targeted integration, and at least 80% of gene integration by treating HSC cells with an aminoquinoline compound, especially with chloroquine and hydroxychloroquine.

The present invention thus encompasses culture media or cultures of HSCs comprising at least 0.005 mM of an aminoquinoline compound and preferably between 0.005 and 1 mM. Such culture media or cultures comprising preferably between 0.01 and 0.5 mM, and more preferably between 0.01 and 0.1 mM chloroquine and/or hydroxychloroquine.

As used herein, the term “hematopoietic stem cells” (or “HSC”) refer to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSC are CD34−. In addition, HSC also refer to long term repopulating HSC (LT-HSC) and short-term repopulating HSC (ST-HSC). LT-HSC and ST-HSC are differentiated, based on functional potential and on cell surface marker expression. For example, in some embodiments, the present invention is preferentially performed on populations of human HSCs comprising long term repopulating HSC (LT-HSC), which express surface markers such as CD34+, CD38−, CD45RA−, CD90+, CD49F+, CD133+ and lin-(negative for mature lineage markers including CD2, CD3, CD4, CD7, CD8, CD10, CD11B, CD19, CD20, CD56, CD235A). Based on studies performed in mice, bone marrow LT-HSC are CD34−, SCA-1+, C-kit+, CD135−, Slamfl/CD150+, CD48-, and lin− (negative for mature lineage markers including Ter119, CD11b, Gr1, CD3, CD4, CD8, B220, IL7ra), whereas ST-HSC are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSC are less quiescent (i.e., more active) and more proliferative than LT-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSC can be used in any of the methods described herein. In some embodiments, ST-HSC are useful because they are highly proliferative and thus, can more quickly give rise to differentiated progeny.

Also the present invention aims to particularly favour homologous recombination events in populations in LT-HSC cells, thereby enriching populations of gene edited cells, which are CD34+, CD38−, CD45RA−, CD90+, CD49F+ and/or CD133+, so as to optimize stem cells engraftment into patients [Psatha, N. et al. (2016) Optimizing autologous cell grafts to improve stem cell gene therapy. Exp Hematol. 44(7): 528-539].

Hematopoietic stem cells (HSCs) can be isolated from bone marrow or by apheresis and be modified ex-vivo and transferred back to the recipient to produce functional, terminally-differentiated cells. As per the methods of the present invention gene correction or gene transfer can be performed in HSCs or in the (differentiated) blood cell types as listed and illustrated in FIG. 7.

The present invention also contemplates combining an aminiquinoline compound as referred to herein with molecules facilitating HSCs expansion such as Nicotinamide (NAM), and cytokines [Peled T, et al. (2012) Nicotinamide, a SIRT1 inhibitor, inhibits differentiation and facilitates expansion of hematopoietic progenitor cells with enhanced bone marrow homing and engraftment. Exp Hematol. 2012; 40:342-355] or with Copper chelation-based expansion techniques using tetraethylenepentamine (TEPA) [Peled T, et al. (2004) Linear polyamine copper chelator tetraethylenepentamine augments long-term ex vivo expansion of cord blood-derived CD34+ cells and increases their engraftment potential in NOD/SCID mice. Exp Hematol. 32:547-55.], as well as StemRegenin 1 (SR1), a purine derivative and aryl hydrocarbon receptor antagonist that reversibly promotes the CD34+ cell expansion by blocking HSC differentiation, UNC0638, UM729 and its more potent analog UM171, that act independently of AhR pathway, as small molecules with the ability to expand SRCs derived from human CD34+CD45RA-mobilized PB cells [Fares I, et al. (2014) Pyrimidoindole derivatives are agonists of human hematopoietic stem cell self-renewal. Science. 345:1509-1512]. In further examples, the exogenous nucleic acid template can comprise sequences for endogenous expression or allele replacement of defective genes such as HBB, STAT3, ADPS1, RAG1, IL2RG, ADA, WAS, Gp91phox, CD18, DCLRE1C, FANCA, ARSA, ABCD1, IDUA, IDS, ARSB, GUSB, ABCD1, GALC, ARSA, PSAP, GBA, FUCA1, MAN2B1, AGA, ASAH1, HEXA, GAA, SMPD1, LIPA and CDKL5, which are known to be involved in inherited pathologies.

In preferred embodiments, the present invention is a method for correcting HBB deficient gene in HSCs by gene targeted integration, wherein a mutated allele of HBB causing sickle cell anemia is reverted to the wild type HBB sequence by treating the cells with an aminoquinoline compound along with a gene editing reagent. Preferred gene editing reagents, preferably a TALE-nuclease, examples of targeted gene sequences and nucleic acid templates are detailed in WO2019185920, which are incorporated by reference.

In further preferred embodiments, the present invention is a method for expressing in HSCs a gene that is deficient in a lysosomal storage disease (LSD) by gene targeted integration of the functional version of said gene with an aminoquinoline compound along with a gene editing reagent specifically targeting specific loci (see below). Examples of such LSDs include Type I Gaucher disease, Fabry disease, Niemann-Pick B disease, Pompe disease, MPS IS, IH/S, IV and VI [Mark S. Sands, et al. (2006) Gene therapy for lysosomal storage diseases, Molecular Therapy, 13(5):839-849]. The genes involved in these inherited disease, which can be complemented according to the invention, are recapitulated in Table 1 below.

TABLE 1
Diseases and transgenes for their treatment.

| | | Functional |
|---|---|---|
| | | polypeptide |
| Disease | Transgene | sequence |
| Mucopolysaccharidosis | IDUA | SEQ ID NO: 1 |
| Type I (Scheie, Hurler- | | |
| Scheie or Hurler | | |
| syndrome) | | |
| Mucopolysaccharidosis | IDS | SEQ ID NO: 2 |
| Type II (Hunter | | |
| syndrome) | | |
| Mucopolysaccharidosis | ARSB | SEQ ID NO: 3 |
| Type VI (Maroteaux- | | |
| Lamy syndrome) | | |
| Mucopolysaccharidosis | GUSB | SEQ ID NO: 4 |
| Type VII (Sly disease) | | |
| X-linked | ABCD1 | SEQ ID NO: 5 |
| Adrenoleukodystrophy | | |
| Globoid Cell | GALC | SEQ ID NO: 6 |
| Leukodystrophy | | |
| (Krabbe disease) | | |
| Metachromatic | ARSA | SEQ ID NO: 7 |
| Leukodystrophy | | |
| Metachromatic | PSAP | SEQ ID NO: 8 |
| Leukodystrophy | | |
| Gaucher disease | GBA | SEQ ID NO: 9 |
| Fucosidosis | FUCA1 | SEQ ID NO: 10 |
| Alpha-mannosidosis | MAN2B1 | SEQ ID NO: 11 |
| Aspartylglucosaminuria | AGA | SEQ ID NO: 12 |
| Farber's disease | ASAH1 | SEQ ID NO: 13 |
| Tay-Sachs disease | HEXA | SEQ ID NO: 14 |
| Pompe disease | GAA | SEQ ID NO: 15 |
| Niemann Pick disease | SMPD1 | SEQ ID NO: 16 |
| Wolman disease | LIPA | SEQ ID NO: 17 |
| CDKL5-deficiency | CDKL5 | SEQ ID NO: 18 |
| related diseases | | |
| (e.g., Early infantile | | |
| epileptic | | |
| encephalopathy (EIEE) | | |
| disease, Atypical Rett | | |
| syndrome, CDKL5- | | |
| related epileptic | | |
| encephalopathy | | |
| disease, or West | | |
| syndrome disease) | | |
| Sickle Cell Anemia | HBB | SEQ ID NO: 19 |
| (SCA) | | |
| X-linked hyper- | CD40L | SEQ ID NO: 20 |
| immunoglobulin M | | |
| syndrome | | |
| Severe obesity | ADCY3 | SEQ ID NO: 21 |
| | BDNF | SEQ ID NO: 22 |
| | KSR2 | SEQ ID NO: 23 |
| | LEP | SEQ ID NO: 24 |

In another aspect, the invention provides methods to obtain isolated HSC or iPS cells which have a transgene integrated at a locus selected from loci highly expressed in microglial cells selected from the group consisting of CCR5, AAVS1, TMEM119, S100A9, CD11B, B2m, Cx3cr1, MERTK, CD164, TIr4, TIr7, Cd14, Fcgr1a, Fcgr3a, TBXAS1, DOK3, ABCA1, TMEM195, MR1, CSF3R, FGD4, TSPAN14, TGFBRI, CCR5, GPR34, SERPINE2, SLCO2B1, P2ryl2, Olfml3, P2ryl3, Hexb, Rhob, Jun, Rab3iI1, CcI2, Fcrls, Scoc, Siglech, Slc2a5, Lrrc3, Plxdc2, Usp2, Ctsf, Cttnbp2nl, Atp8a2, Lgmn, Mafb, Egr1, Bhlhe41, Hpgds, Ctsd, Hspa1a, Lag3, Csf1r, Adamts1, F11r, GoIm1, Nuak1, Crybb1, Ltc4s, Sgce, Pla2g15, Ccl3l1, Abhd12, Ang, Ophn1, Sparc, Pros1, P2ry6, Lair1, II1a, Epb41I2, Adora3, Rilpl1, Pmepa1, CcI13, Pde3b, Scamp5, Ppp1r9a, Tjp1, Ak1, B4galt4, Gtf2h2, Trem2, Ckb, Acp2, Pon3, Agmo, Tnfrsf17, Fscn1, St3gal6, Adap2, Ccl4, Entpd1, Tmem86a, Kctd12, Dst, Ctsl2, Abcc3, Pdgfb, Pald1, Tubgcp5, Rapgef5, Stab1, Lacc1, Tmc7, Nrip1, Kcnd1, Tmem206, Hps4, Dagla, ExtI3, Mlph, Arhgap22, Cxxc5, P4ha1, Cysltr1, Fgd2, Kcnk13, Gbgt1, C18orf1, Cadm1, Bco2, Adrb1, C3ar1, Large, Leprel1, Liph, Upk1b, P2rx7, Slc46a1, Ebf3, Ppp1r15a, Il10ra, Rasgrp3, Fos, Tppp, Slc24a3, Havcr2, Nav2, Apbb2, Clstn1, Blnk, Gnaq, Ptprm, Frmd4a, Cd86, Tnfrsf11a, Spint1, Ppm1l, Tgfbr2, Cmklr1, TIr6, Gas6, Hist1h2ab, Atf3, Acvr1, Abi3, Lrp12, Ttc28, Plxna4, Adamts16, Rgs1, Icam1, Snx24, Ly96, Dnajb4, and Ppfia4.

Among the above loci, CCR5, AAVS1, STAT3, ADPS1, RAG1, TMEM119, MERTK, CD164, TLR7, CD14, FCGR3A (CD16), TBXAS1, DOK3, ABCA1, TMEM195, TLR4, MR1, FCGR1A (CD64), CSF3R, FGD4, TSPAN14, CXCR3, CD11B, S100A9, B2M. IL2RG, ADA, WAS, Gp91phox, CD18, DCLRE1C, FANCA, ARSA, ABCD1 and IDUA are preferred in the context of transforming HSCs as per the present invention.

In some embodiments, the exogenous sequence is inserted at a locus selected from CD25, CD69 or one listed in Table 3 (list of gene loci upregulated in tumor exhausted infiltrating lymphocytes), or Table 4 (list of gene loci upregulated in hypoxic tumor conditions).

TABLE 3
List of gene loci upregulated in tumor exhausted infiltrating
lymphocytes useful for gene integration of exogenous
coding sequences as per the present invention.

| | Uniprot ID |
|---|---|
| | (www.uniprot.org) |
| Gene names | (human) |
| GM-CSF | P04141 |
| CXCL13 | O43927 |
| TNFRSF1B | P20333 |
| RGS2 | P41220 |
| TIGIT | Q495A1 |
| CD27 | P26842 |
| TNFRSF9 | Q12933 |
| SLA | Q13239 |
| INPP5F | Q01968 |
| XCL2 | Q9UBD3 |
| HLA-DMA | P28067 |
| FAM3C | Q92520 |
| WARS | P23381 |
| EIF3L | Q9Y262 |
| KCNK5 | O95279 |
| TMBIM6 | P55061 |
| CD200 | P41217 |
| C3H7A | O60880 |
| SH2D1A | O60880 |
| ATP1B3 | P54709 |
| THADA | Q6YHU6 |
| PARK7 | Q99497 |
| EGR2 | P11161 |
| FDFT1 | P37268 |
| CRTAM | O95727 |
| IFI16 | Q16666 |

TABLE 4
List of gene loci upregulated in hypoxic tumor conditions
useful for gene integration of exogenous coding
sequences as per the present invention.

| | Strategy | |
|---|---|---|
| | (KO—knock out; | |
| Gene names | KI—knock in) | |
| CTLA-4 | KO/KI | Target shown to be upregulated in |
| LAG-3 (CD223) | KO/KI | T-cells upon hypoxia exposure and |
| PD1 | KO/KI | T cell exhaustion |
| 4-1BB (CD137) | KI | |
| GITR | KI | |
| OX40 | KI | |
| IL10 | KO/KI | |
| ABCB1 | KI | Loci which expression is under |
| ABCG2 | KI | HIF-1 (Uniprot Q16665) |
| ADM | KI | dependency. |
| ADRA1B | KI | |
| AK3 | KI | |
| ALDOA | KI | |
| BHLHB2 | KI | |
| BHLHB3 | KI | |
| BNIP3 | KI | |
| BNIP3L | KI | |
| CA9 | KI | |
| CCNG2 | KI | |
| CD99 | KI | |
| CDKN1A | KI | |
| CITED2 | KI | |
| COL5A1 | KI | |
| CP | KI | |
| CTGF | KI | |
| CTSD | KI | |
| CXCL12 | KI | |
| CXCR4 | KI | |
| CYP2S1 | KI | |
| DDIT4 | KI | |
| DEC1 | KI | |
| EDN1 | KI | |
| EGLN1 | KI | |
| EGLN3 | KI | |
| ENG | KI | |
| ENO1 | KI | |
| EPO | KI | |
| ETS1 | KI | |
| FECH | KI | |
| FN1 | KI | |
| FURIN | KI | |
| GAPDH | KI | |
| GPI | KI | |
| GPX3 | KI | |
| HK1 | KI | |
| HK2 | KI | |
| HMOX1 | KI | |
| HSP90B1 | KI | |
| ID2 | KI | |
| IGF2 | KI | |
| IGFBP1 | KI | |
| IGFBP2 | KI | |
| IGFBP3 | KI | |
| ITGB2 | KI | |
| KRT14 | KI | |
| KRT18 | KI | |
| KRT19 | KI | |
| LDHA | KI | |
| LEP | KI | |
| LOX | KI | |
| LRP1 | KI | |
| MCL1 | KI | |
| MET | KI | |
| MMP14 | KI | |
| MMP2 | KI | |
| MXI1 | KI | |
| NOS2A | KI | |
| NOS3 | KI | |
| NPM1 | KI | |
| NR4A1 | KI | |
| NT5E | KI | |
| PDGFA | KI | |
| PDK1 | KI | |
| PFKFB3 | KI | |
| PFKL | KI | |
| PGK1 | KI | |
| PH-4 | KI | |
| PKM2 | KI | |
| PLAUR | KI | |
| PMAIP1 | KI | |
| PPP5C | KI | |
| PROK1 | KI | |
| SERPINE1 | KI | |
| SLC2A1 | KI | |
| TERT | KI | |
| TF | KI | |
| TFF3 | KI | |
| TFRC | KI | |
| TGFA | KI | |
| TGFB3 | KI | |
| TGM2 | KI | |
| TPI1 | KI | |
| VEGFA | KI | |
| VIM | KI | |
| TMEM45A | KI | |
| AKAP12 | KI | |
| SEC24A | KI | |
| ANKRD37 | KI | |
| RSBN1 | KI | |
| GOPC | KI | |
| SAMD12 | KI | |
| CRKL | KI | |
| EDEM3 | KI | |
| TRIM9 | KI | |
| GOSR2 | KI | |
| MIF | KI | |
| ASPH | KI | |
| WDR33 | KI | |
| DHX40 | KI | |
| KLF10 | KI | |
| R3HDM1 | KI | |
| RARA | KI | |
| LOC162073 | KI | |
| PGRMC2 | KI | |
| ZWILCH | KI | |
| TPCN1 | KI | |
| WSB1 | KI | |
| SPAG4 | KI | |
| GYS1 | KI | |
| RRP9 | KI | |
| SLC25A28 | KI | |
| NTRK2 | KI | |
| NARF | KI | |
| ASCC1 | KI | |
| UFM1 | KI | |
| TXNIP | KI | |
| MGAT2 | KI | |
| VDAC1 | KI | |
| SEC61G | KI | |
| SRP19 | KI | |
| JMJD2C | KI | |
| SNRPD1 | KI | |
| RASSF4 | KI | |

In some embodiments, multiples copies of the transgene are integrated at the same locus separated by 2A self-cleaving peptide sequences. In some embodiments, the therapeutic gene product will be under the regulatory control of the target locus and promote expression in hematopoietic cells and in particular the microglial cells. The modified cells can subsequently be returned to the patient through adoptive cell transfer or autologous HSC transplantation. This process will deliver the therapeutic gene product systemically to treat the body but also locally in the brain to treat symptoms of brain disease by cross correction.

Preferred loci for targeted gene integration are CCR5, AAVS1, TMEM119, CD11B, B2m, CX3CR1 and S100A9, especially in view of producing cells expressing a therapeutic transgene in HSCs that can differentiate into microglial cells, especially to prevent or treat inherited metabolic disorders.

As an independent embodiment, is provided a method for integrating an exogenous coding sequence into an endogenous intronic genomic region, which allows integration of said exogenous coding sequence preferably between the first and second endogenous exons of said genomic region. In some embodiments, this method has the advantage to preserve stemness of HSCs and their ability to differentiate into various myeloid cells.

The present invention contributes to treating many inherited disease by integration of a transgene into therapeutic cells. In preferred embodiments, the transgene comprises:
HBB for treating Sickle Cell Anemia (SCA);
CD40L for treating X-linked hyper-immunoglobulin M syndrome;
IDUA for treating Mucopolysaccharidosis Type I (Scheie, Hurler-Scheie or Hurler syndrome),
IDS for treating Mucopolysaccharidosis Type II (Hunter),
ARSB for treating Mucopolysaccharidosis Type VI (Maroteaux-Lamy),
GUSB for treating Mucopolysaccharidosis Type VII (Sly),
ABCD1 for treating X-linked Adrenoleukodystrophy,
GALC for treating Globoid Cell Leukodystrophy (Krabbe),
ARSA for treating Metachromatic Leukodystrophy,
GBA for treating Gaucher Disease,
FUCA1 for treating Fucosidosis,
MAN2B1 for treating Alpha-mannosidosis,
AGA for treating Aspartylglucosaminuria,
ASAH1 for treating Farber Disease,
HEXA for treating Tay-Sachs Disease,
GAA for treating Pompe Disease,
SMPD1 for treating Niemann Pick Disease,
DMD for treating Duchenne muscular dystrophy
LIPA for treating Wolman Syndrome,
CDKL5 for treating CDKL5-deficiency related disease, or
ADCY3, BDNF, KSR2, LEP for treating severe obesity.

In some other embodiments, the present methods can be used to integrate a transgene encoding a chimeric antigen receptor CAR or a recombinant TCR in immune cells for producing therapeutic cells, such as T-cells or NK cells, for the treatment of cancer or infection as described for instance in WO2013176915.

Transgenes in T-cells can be advantageously integrated at specific loci such as TCR, GM-CSF, B2M, GCN2, PD1, CTLA4, TIM3, LAG3, DCK, HPRT, GGH, GR, CD52, TGFb, TGFbR, IL-10, IL-10R and/or CISH, which have been previously described to improve therapeutic potency and/or safety of engineered T-cells, especially CAR T-cells (see WO2018073391 and Table 2).

One particular focus of the present invention is to perform gene inactivation in primary immune cells at a locus, by integrating exogenous coding sequence at said locus, the expression of which improves the therapeutic potential of said engineered cells. Examples of relevant exogenous coding sequences that can be inserted according to the invention have been presented above in connection with their positive effects on the therapeutic potential of the cells. Here below are presented the endogenous gene that are preferably targeted by gene targeted insertion and the advantages associated with their inactivation.

According to a preferred aspect of the invention, the insertion of the coding sequence has the effect of reducing or preventing the expression of genes involved into self and non-self recognition to reduce host versus graft disease (GVHD) reaction or immune rejection upon introduction of the allogeneic cells into a recipient patient. For instance, one of the sequence-specific reagents used in the method can reduce or prevent the expression of TCR in primary T-cells, such as the genes encoding TCR-alpha or TCR-beta.

As another preferred aspect, one gene editing step is to reduce or prevent the expression of the β2m protein and/or another protein involved in its regulation such as CIITA (Uniprot #P33076) or in MHC recognition, such as HLA proteins. This permits the engineered immune cells to be less alloreactive when infused into patients.

By “allogeneic therapeutic use” is meant that the cells originate from a donor in view of being infused into patients having a different haplotype. Indeed, the present invention provides with an efficient method for obtaining primary cells, which can be gene edited in various gene loci involved into host-graft interaction and recognition.

Other loci may also be edited in view of improving the activity, the persistence of the therapeutic activity of the engineered primary cells as detailed here after:

According to a preferred aspect of the invention, the inserted exogenous coding sequence has the effect of reducing or preventing the expression of a protein involved in immune cells inhibitory pathways, in particular those referred to in the literature as “immune checkpoint” [Pardoll, D. M. (2012) The blockade of immune checkpoints in cancer immunotherapy, Nature Reviews Cancer, 12:252-264]. In the sense of the present invention, “immune cells inhibitory pathways” means any gene expression in immune cells that leads to a reduction of the cytotoxic activity of the lymphocytes towards malignant or infected cells. This can be for instance a gene involved into the expression of FOXP3, which is known to drive the activity of Tregs upon T cells (moderating T-cell activity).

“Immune checkpoints” are molecules in the immune system that either turn up a signal (co-stimulatory molecules) or turn down a signal of activation of an immune cell. As per the present invention, immune checkpoints more particularly designate surface proteins involved in the ligand—receptor interactions between T cells and antigen-presenting cells (APCs) that regulate the T cell response to antigen (which is mediated by peptide—major histocompatibility complex (MHC) molecule complexes that are recognized by the T cell receptor (TCR)). These interactions can occur at the initiation of T cell responses in lymph nodes (where the major APCs are dendritic cells) or in peripheral tissues or tumours (where effector responses are regulated). One important family of membrane-bound ligands that bind both co-stimulatory and inhibitory receptors is the B7 family. All of the B7 family members and their known ligands belong to the immunoglobulin superfamily. Many of the receptors for more recently identified B7 family members have not yet been identified. Tumour necrosis factor (TNF) family members that bind to cognate TNF receptor family molecules represent a second family of regulatory ligand—receptor pairs. These receptors predominantly deliver co-stimulatory signals when engaged by their cognate ligands. Another major category of signals that regulate the activation of T cells comes from soluble cytokines in the microenvironment. In other cases, activated T cells upregulate ligands, such as CD40L, that engage cognate receptors on APCs. A2aR, adenosine A2a receptor; B7RP1, B7-related protein 1; BTLA, B and T lymphocyte attenuator; GAL9, galectin 9; HVEM, herpesvirus entry mediator; ICOS, inducible T cell co-stimulator; IL, interleukin; KIR, killer cell immunoglobulin-like receptor; LAG3, lymphocyte activation gene 3; PD1, programmed cell death protein 1; PDL, PD1 ligand; TGFβ, transforming growth factor-β; TIM3, T cell membrane protein 3.

Examples of further endogenous genes, which expression could be reduced or suppressed to turn-up activation in the engineered immune cells according the present invention are listed in Table 5.

TABLE 5
List of genes involved into immune cells inhibitory pathways

| | | Genes that can be |
|---|---|---|
| Pathway | | inactivated In the pathway |
| Co-inhibitory | CTLA4 (CD152) | CTLA4, PPP2CA, |
| receptors | | PPP2CB, PTPN6, PTPN22 |
| | PDCD1 (PD-1, CD279) | PDCD1 |
| | CD223 (lag3) | LAG3 |
| | HAVCR2 (tim3) | HAVCR2 |
| | BTLA(cd272) | BTLA |
| | CD160(by55) | CD160 |
| | IgSF family | TIGIT |
| | | CD96 |
| | | CRTAM |
| | LAIR1(cd305) | LAIR1 |
| | SIGLECs | SIGLEC7 |
| | | SIGLEC9 |
| | CD244(2b4) | CD244 |
| Death receptors | TRAIL | TNFRSF10B, TNFRSF10A, |
| | | CASP8, CASP10, |
| | | CASP3, CASP6, CASP7 |
| | FAS | FADD, FAS |
| Cytokine | TGF-beta signaling | TGFBRII, TGFBRI, |
| signalling | | SMAD2, SMAD3, SMAD4, |
| | | SMAD10, SKI, SKIL, TGIF1 |
| | IL10 signalling | IL10RA, IL10RB, HMOX2 |
| | IL6 signalling | IL6R, IL6ST |
| Prevention of | | CSK, PAG1 |
| TCR signalling | | SIT1 |
| Induced Treg | induced Treg | FOXP3 |
| Transcription | transcription factors | PRDM1 |
| factors | controlling exhaustion | BATF |
| controlling | | |
| exhaustion | | |
| Hypoxia | iNOS induced | GUCY1A2, GUCY1A3, |
| mediated | guanylated cyclase | GUCY1B2, GUCY1B3 |
| tolerance | | |

For instance, the inserted exogenous coding sequence(s) can have the effect of reducing or preventing the expression, by the engineered immune cell of at least one protein selected from PD1 (Uniprot Q15116), CTLA4 (Uniprot P16410), PPP2CA (Uniprot P67775), PPP2CB (Uniprot P62714), PTPN6 (Uniprot P29350), PTPN22 (Uniprot Q9Y2R2), LAG3 (Uniprot P18627), HAVCR2 (Uniprot Q8TDQ0), BTLA (Uniprot Q7Z6A9), CD160 (Uniprot 095971), TIGIT (Uniprot Q495A1), CD96 (Uniprot P40200), CRTAM (Uniprot 095727), LAIR1 (Uniprot Q6GTX8), SIGLEC7 (Uniprot Q9Y286), SIGLEC9 (Uniprot Q9Y336), CD244 (Uniprot Q9BZW8), TNFRSF10B (Uniprot 014763), TNFRSF10A (Uniprot 000220), CASP8 (Uniprot Q14790), CASP10 (Uniprot Q92851), CASP3 (Uniprot P42574), CASP6 (Uniprot P55212), CASP7 (Uniprot P55210), FADD (Uniprot Q13158), FAS (Uniprot P25445), TGFBRII (Uniprot P37173), TGFRBRI (Uniprot Q15582), SMAD2 (Uniprot Q15796), SMAD3 (Uniprot P84022), SMAD4 (Uniprot Q13485), SMAD10 (Uniprot B7ZSB5), SKI (Uniprot P12755), SKIL (Uniprot P12757), TGIF1 (Uniprot Q15583), MORA (Uniprot Q13651), IL10RB (Uniprot Q08334), HMOX2 (Uniprot P30519), IL6R (Uniprot P08887), IL6ST (Uniprot P40189), EIF2AK4 (Uniprot Q9P2K8), CSK (Uniprot P41240), PAG1 (Uniprot Q9NWQ8), SIT1 (Uniprot Q9Y3P8), FOXP3 (Uniprot Q9BZS1), PRDM1 (Uniprot Q60636), BATF (Uniprot Q16520), GUCY1A2 (Uniprot P33402), GUCY1A3 (Uniprot Q02108), GUCY1B2 (Uniprot Q8BXH3) and GUCY1B3 (Uniprot Q02153). The gene editing introduced in the genes encoding the above proteins is preferably combined with an inactivation of TCR in CAR T cells.

According to another aspect of the invention, the inserted exogenous coding sequence has the effect of reducing or preventing the expression of genes encoding or positively regulating suppressive cytokines or metabolites or receptors thereof, in particular TGFbeta (Uniprot:P01137), TGFbR (Uniprot:P37173), IL10 (Uniprot:P22301), IL10R (Uniprot: Q13651 and/or Q08334), A2aR (Uniprot: P29274), GCN2 (Uniprot: P15442) and PRDM1 (Uniprot: 075626).

Preference is given to engineered immune cells in which a sequence encoding IL-2, IL-12 or IL-15 replaces the sequence of at least one of the above endogenous genes.

According to another aspect of the present method, the transgene sequence can have the effect of reducing or preventing the expression of a gene responsible for the sensitivity of the immune cells to compounds used in standard of care treatments for cancer or infection, such as drugs purine nucleotide analogs (PNA) or 6-Mercaptopurine (6MP) and 6 thio-guanine (6TG) commonly used in chemotherapy. Reducing or inactivating the genes involved into the mode of action of such compounds (referred to as “drug sensitizing genes”) improves the resistance of the immune cells to same.

Examples of drug sensitizing gene are those encoding DCK (Uniprot P27707) with respect to the activity of PNA, such a clorofarabine et fludarabine, HPRT (Uniprot P00492) with respect to the activity of purine antimetabolites such as 6MP and 6TG, and GGH (Uniprot Q92820) with respect to the activity of antifolate drugs, in particular methotrexate.

This enables the cells to be used after or in combination with conventional anti-cancer chemotherapies.

According to another aspect of the present invention, the inserted exogenous coding sequence has the effect of reducing or preventing the expression of receptors or proteins, which are drug targets, making said cells resistant to immune-depletion drug treatments. Such target can be glucocorticoids receptors or antigens, to make the engineered immune cells resistant to glucocorticoids or immune depletion treatments using antibodies such as Alemtuzumab, which is used to deplete CD52 positive immune cells in many cancer treatments.

Also, the method of the invention can comprise gene targeted insertion in endogenous gene(s) encoding or regulating the expression of CD52 (Uniprot P31358) and/or GR (Glucocorticoids receptor also referred to as NR3C1-Uniprot P04150).

Transgenes in NK cells can be advantageously integrated at specific loci, such as TGF-β receptor, Cbl-B, A2A receptor, KLRD1, LIR1/ILT2, KIRs, AhR, Tim-3, Tyro-3, GCN2, CD94, CD74, cyclophilin A, TBL1XR1, HPRT, dCK, CDS, beta2M and PD-1, which inactivations have been described to improve therapeutic potency and/or safety of engineered NK-cells as referred to for instance in WO2017001572.

In some other embodiments, the nucleic acid template in the present invention can comprise gene sequence to improve cell's functionality or confer cells resistance to drugs or to particular tumor environment conditions. As an example, gene sequences such as encoding decoys of HLAE or HLAG, viral evasins or fragment(s) comprising an epitope thereof, such as from UL16 (also called ULBP1-Uniprot ref.: #Q9BZM6) can be integrated into T-cells as described in WO2019076486 to escape NK cells destruction. As another example, gene sequences encoding soluble polypeptides that interfere with pro-inflammatory cytokine pathways, such as IL1RA, sgp130Fc, IL18BP, respectively interfering with IL1, IL6 and IL18, can be integrated in therapeutic cells to lower the risk of inducing cytokine release syndrome (CRS) as described in WO2019076489. As a further example, gene sequence encoding ALDH, MGMT, MTX, GST, cytidine deaminase, IL2 receptor (CD25), IL15-2A-IL15 receptor, IFN gamma, Lysteria P60, TNF and IL12-α can be integrated into NK cells to improve their functionality as described for instance in WO2017001572. Many other examples of transgenes can be found including those express siRNA or shRNA to inhibit the expression of immune regulatory or MHC genes, such as B2M in CAR T-cells, as described in McCreedy, B. J. et al. [Off the shelf T cell therapies for hematologic malignancies (2018) Best Practice & Research Clinical Haematology, 31 (2): 166-175].

The method of the present invention described above allows producing engineered primary immune cells within a limited time frame of about 15 to 30 days, preferably between 15 and 20 days, and most preferably between 18 and 20 days so that they keep their full immune therapeutic potential, especially with respect to their cytotoxic activity.

These cells form a population of cells, which preferably originate from a single donor or patient. These populations of cells can be expanded under closed culture recipients to comply with highest manufacturing practices requirements and can be frozen prior to infusion into a patient, thereby providing “off the shelf” or “ready to use” therapeutic compositions.

As per the present invention, a significant number of cells originating from the same Leukapheresis can be obtained, which is critical to obtain sufficient doses for treating a patient. Although variations between populations of cells originating from various donors may be observed, the number of immune cells procured by a leukapheresis is generally about from 10⁸ to 10¹⁰ cells of PBMC. PBMC comprises several types of cells: granulocytes, monocytes and lymphocytes, among which from 30 to 60% of T-cells, which generally represents between 10⁸ to 10⁹ of primary T-cells from one donor. The method of the present invention generally ends up with a population of engineered cells that reaches generally more than about 10⁸ T-cells, more generally more than about 10⁹ T-cells, even more generally more than about 10¹⁰ T-cells, and usually more than 10¹¹ T-cells.

The invention is thus more particularly drawn to a therapeutically effective population of primary immune cells, wherein at least 30%, preferably 50%, more preferably 80% of the cells in said population have been modified according to any one the methods described herein. Said therapeutically effective population of primary immune cells, as per the present invention, comprises immune cells that have integrated at least one exogenous genetic sequence.

Such compositions or populations of cells can therefore be used as medicaments; especially for treating cancer, particularly for the treatment of lymphoma, but also for solid tumors such as melanomas, neuroblastomas, gliomas or carcinomas such as lung, breast, colon, prostate or ovary tumors in a patient in need thereof.

The invention is more particularly drawn to populations of primary TCR negative T-cells originating from a single donor, wherein at least 20%, preferably 30%, more preferably 50% of the cells in said population have been modified using sequence-specific reagents in at least two, preferably three different loci.

The treatments involving the engineered primary immune cells according to the present invention can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

In another embodiment, said isolated cell according to the invention or cell line derived from said isolated cell can be used for the treatment of liquid tumors, and preferably of T-cell acute lymphoblastic leukemia.

The treatment with the engineered immune cells according to the invention may be in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment can be administrated into patients undergoing an immunosuppressive treatment. Indeed, the present invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient.

When CARs are expressed in the immune cells or populations of immune cells according to the present invention, the preferred CARs are those targeting at least one antigen selected from CD22, CD38, CD123, CS1, HSP70, ROR1, GD3, and CLL1.

The engineered immune cells according to the present invention endowed with a CAR or a modified TCR targeting CD22 are preferably used for treating leukemia, such as acute lymphoblastic leukemia (ALL), those with a CAR or a modified TCR targeting CD38 are preferably used for treating leukemia such as T-cell acute lymphoblastic leukemia (T-ALL) or multiple myeloma (MM), those with a CAR or a modified TCR targeting CD123 are preferably used for treating leukemia, such as acute myeloid leukemia (AML), and blastic plasmacytoid dendritic cells neoplasm (BPDCN), those with a CAR or a modified TCR targeting CS1 are preferably used for treating multiple myeloma (MM).

In further embodiment of the present invention, the aminoquinoline compounds used to promote gene targeted integration can be combined with reagents which are known in the art to favor a given gene repair pathways in the cell, referred to herein as “repair pathway reagents”. As shown in FIG. 8, double strand break induced by endonucleases reagents can be repaired by different pathways managed by different key proteins. One objective of the present invention is to stimulate homologous recombination events as far as possible over non-homologous end-joining (NHEJ) pathways or other error prone repair pathways. To this aim, appropriate repair pathway reagents can either inhibit NHEJ pathway, such as compounds like STL127705, NU7441, KU-0060648, NU7026, M3812, E-822, SCR7, RS-1, can act on cell cycle, such as Wortmanin, Aphidicolin, mimosin thymidine, Hydroxy urea (HU), Nocodazole, ABT-751, XL413, or induce targeted integration increase by so far unknown mechanisms, such as L755507, Brefeldin and Resveratrol. Other preferred “repair pathway reagents” are inhibitors of lig4, xrcc4, Ku70, Ku80, DNA-PKcs, which can be shRNA or siRNA transfected or expressed into the cell directed against lig4, xrcc4, Ku70, Ku80, DNA-PKcs transcripts. In further embodiments, the methods of the present invention further comprise expressing into the cells a nucleic acid encoding Rad51, Rad52, E4orf6/7, dominant-negative p53 mutant protein (GSE56), inhibitor of 53PB1 and/or dominant-negative 53BP1. Such polynucleotides encoding Rad51, Rad52, E4orf6/7, dominant-negative p53 mutant protein (GSE56), inhibitor of 53PB1 and/or dominant-negative 53BP1 can be transfected in the same time as the gene editing reagents and/or the nucleic acid template [Canny M. D., et al. (2018) Inhibition of 53BP1 favors homology-dependent DNA repair and increases CRISPR-Cas9 genome-editing efficiency. Nat Biotechnol. 36(1): 95-102], [Paulsen B. S., et al. (2017) Ectopic expression of RAD52 and dn53BP1 improves homology-directed repair during CRISPR-Cas9 genome editing. Nat Biomed 1(11):878-888], [Schiroli, G. et al. (2019) Precise Gene Editing Preserves Hematopoietic Stem Cell Function following Transient p53-Mediated DNA Damage Response Cell Stem Cell 24:551-565].

The present invention is also drawn to compositions, especially therapeutic compositions, kits, or nanoparticles comprising at least an aminoquinioline compound(s), to perform any of the steps of the methods previously described.

More particularly, it encompasses compositions, kits, or nanoparticles for transfecting cells comprising:
(a) an aminoquinoline compound (as claimed before), and
(b) a nucleic acid template to be integrated into the genome of a cell at a selected locus, and/or
(c) a sequence specific nuclease reagent.

Such compositions, kits, or nanoparticles can further comprise at least one “repair pathway reagent” to stimulate homologous recombination selected from the compounds: STL127705, NU7441, KU-0060648, NU7026, M3812, E-822, SCR7, RS-1, Wortmanin, Aphidicolin, mimosin thymidine, Hydroxy urea (HU), Nocodazole, ABT-751, XL413 L755507, Brefeldin and Resveratrol.

Compositions, kits, or nanoparticles according to the present invention can further comprise inhibitors of lig4, xrcc4, Ku70, Ku80, DNA-PKcs, preferably shRNA or siRNA.

Compositions, kits, or nanoparticles according to the invention can further comprise nucleic acids expressing Rad51, Rad52, E4orf6/7, dominant-negative p53 mutant protein (GSE56), inhibitor of 53PB1 and/or dominant-negative 53BP1.

Successful clinical outcome of HSC transplantation and gene therapy depends not only on high cell numbers but also on efficient homing and engraftment of cells to the bone marrow or cell adhesion to the bone marrow stroma. Also, the present invention combines an aminiquinoline compound treatment as referred to herein with molecules facilitating HSCs homing/engraftment, such as ProstaglandinE2 (PGE2) [Cutler C, et al. (2013) Prostaglandin-modulated umbilical cord blood hematopoietic stem cell transplantation. Blood. 122:3074-81], and/or inhibitors of Dipeptidylpeptidase 4 (DPP4 or CD26) on their surface. such as Diprotin A (DipA). In preferred embodiments, treatment with PGE2 and/or DipA is performed as a subsequent step. According to a further embodiment, the invention can couple the step of treating the cells with an aminoquinoline compound and inducing quiescence of the gene edited cells, for instance by using compounds such as Rapamycin and CHIR99021, the later acting as an inhibitor of the enzyme GSK-3. Inducing quiescence upon gene editing step can be obtained by supplementing culture media with 1 to 10 nM Rapamycin (EMD Millipore) and/or 1-10 μM CHIR99021 (EMD Millipore), as shown by Shin et al. [Controlled Cycling and Quiescence Enables Efficient HDR in Engraftment-Enriched Adult Hematopoietic Stem and Progenitor Cells (2020) Cell Reports. 32, 108093]. More particularly, the invention provides treating the cells with compositions or culture media combining an aminoquinoline compound, Rapamycin and/or CHIR99021.

Particular methods and compositions of the invention pertain to assays to assess the specificity of gene editing reagents, such as an oligo capture assay (OCA), in which integration of labelled polynucleotide probes into the genome by said gene editing reagent is stimulated by addition of aminoquinoline compounds in the reaction. Such assays allow to detect on-target and off-target integrations induced by the gene editing reagent. Oligo capture assay (OCA) or other types of nucleic acid capture assays for quantitation of nucleic acids integrated into the genome [Tsai S. Q. et al. (2015) GUIDE-Seq enables genome-wide profiling of off-target cleavage by CRISPR-Cas nucleases. Nat Biotechnol 33(2): 187-197] can be improved by the present invention.

The invention thus provides an oligo capture assay (OCA) method, characterized in that cells are treated with an aminoquinoline compound(s) to increase oligonucleotide markers integration into the genome of said cells.

The present invention can be regarded as a method for non-viral gene delivery of transgene into cells, in particular HSCs, meaning that targeted gene integration can be obtained without integrative viral vectors.

The present invention also pertains to cell cultures and culture media comprising at least 0,001 mM of an aminoquinoline compound as described herein, preferably between and 1 mM, and more preferably between 0.01 et 1 mM. Such cell cultures or media can specifically comprise between 0,005 and 0.05 mM, and more preferably between 0.01 and mM chloroquine or hydroxychloroquine.

The methods and compositions described herein can be used to transform any cell types, especially in view of generating therapeutic cells or cell lines for use in gene therapy. Such methods can be performed ex-vivo, prior to infusing the cells or populations of cells into a recipient organism or patient.

The present invention thus encompasses gene therapy methods comprising the step of administrating, sequentially or in combination: (a) an aminoquinoline compound, (b) a nucleic acid template, and/or/optionally (c) a gene editing reagent.

The invention more specifically aims to provide/develop ex-vivo gene therapy methods for treating any of the pathologies referred to previously comprising the step of contacting a cell sequentially or concomitantly with (1) an aminoquinoline compound, and (2) an exogenous nucleic acid template, and/or/optionally (3) sequence-specific gene editing reagent, preferably an endonuclease or nickase reagent.

Having generally described this invention, a further understanding can be obtained by reference to certain specific examples, which are provided herein for purposes of illustration only, and are not intended to limit the scope of the claimed invention.

### EXAMPLES

### Example 1: Material and Methods

### Cell Culture:

HSC culture: HSCs prepared from mobilized Leukopak (Miltenyi), were thawed and seeded at 0.4×10⁶ cells/ml into expansion media composed of STEM Span II media (cat. #09655, Stemcell Technologies), with 1× final concentrations of CD34+ expansion cocktail (#02691, Stemcell Technologies) and Pen-Strep (#15140-122, Gibco Life Technologies). The cells were incubated at 37° C. and 5% CO₂ for 48 hrs for recovery after thawing before TALEN transfection and AAV transduction.

T cell culture: Cryopreserved human PBMCs was purchased from Allcells. PBMCs were thawed and cultured in in X-vivo-15 media (Lonza Cat #04-418Q), containing 20 ng/ml IL-2 (Miltenyi biotec Cat #130-097-743), and 5% human serum AB (Gemini Cat #H47Y00L) at a density of 2 10⁶/ml overnight before activation. Human T activator TransAct beads (Miltenyi Biotec Cat #130-111-160) were used to activate PBMCs, according to the provider's protocol, to activate T-cells for 3 days.

### Chloroquine and Hydroxychloroquine Solution:

Chloroquine diphosphate (Sigma ref. C6628), Hydroxyhloroquine sulfate (Sigma ref. H9015) (Sigma-Aldrich, Inc., 3050 Spruce Street, St. Louis, Missouri, U.S.A.) were dissolved in ddH₂O to make a 10 mM stock solution. The solution was filtered through 0.2 mM filter to sterilization and aliquoted and stored at −20° C. A fresh aliquot is used for every experiment.

### Repair Template Constructs:

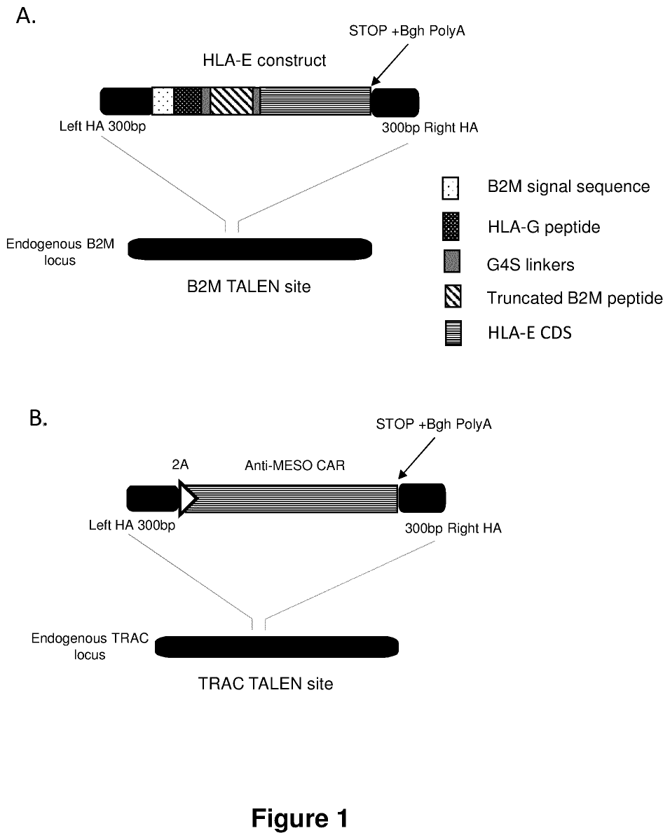

For the B2M locus, AAV6 particles (titer 2.82E13 GC per ml) obtained from Vigene were used to insert an HLA-E repair template into B2M locus (SEQ ID NO. 27). The insert contains a B2M signal sequence (SEQ ID NO. 45), followed by a short HLA-G peptide (SEQ ID NO. 43), a 3×G4S liner (SEQ ID NO. 44), a truncated B2M peptide (SEQ ID NO. 45), a 4×G4S liner (SEQ ID NO. 46), the full length HLA-E coding sequence (SEQ ID NO. 47) followed by BGH poly A sequence (SEQ ID NO. 29. The insert is flanked by 300 bp left (SEQ ID NO. 32) and a right (SEQ ID NO. 33) homology arm of the B2M locus (FIG. 1A).

For the TRAC locus, AAV6 particles (FIG. 1B) were used to insert a polynucleotide encoding for anti-mesothelin CAR (MESO-CAR) at the TRAC locus under TCR promoter dependence. The insert contains a self-cleaving peptide 2A, followed by in frame sequence of the MESO-CAR (SEQ ID NO. 28) and BGH poly A sequence (SEQ ID NO. 29), this insert is flanked by 300 bp left (SEQ ID NO. 34) and a right (SEQ ID NO. 35) homology arm of the TRAC locus (FIG. 1B).

### TALE-Nucleases Reagents:

mRNAs encoding TRAC TALEN (SEQ NO. 36 and 37) and mRNAs encoding B2M TALEN (SEQ NO. 38 and 39) were produced according to previously described protocol (Poirot et al. 2015). The targeted sequences are TCCGTGGCCTTAGCTGTgctcgcgctactcTCTCTTTCTGGCCTGGA (SEQ ID NO. 25) and TTCCTCCTACTCACCATcagcctcctggttatGGTACAGGTAAGAGCAA (SEQ ID NO. 26) for B2M and TRAC TALEN respectively. (TALEN® is a trade name for TALE-nucleases heterodimers designed by Cellectis—8 rue de la Croix Jarry, Paris, France—under license of WO2011072246).

### Electroporation Step for Transfection of the TALE-Nucleases Reagents:

In HSCs, on the day of transfection and transduction, 400 μl of expansion media was prewarmed at 37° C. in a 48 well plate until electroporation. HSCs were harvested, washed once with PBS, then resuspended in 100 μl of High Performance electroporation buffer (#45-0802, BTX) at a concentration of 10×10⁶ cells/ml. The B2M TALEN mRNAs were mixed with cell suspension at 10 μg each TALEN arm per million cells. The cell and mRNA mixtures were electroporated on BTX PulseAgile, using the program shown in Table 6. The HSCs were transferred into the prewarmed expansion media to give a final concentration of 2×10⁶ cells/ml.

TABLE 6
BTX PulseAgile settings for HSCs

| Settings | Group1 | Group2 | Group3 |
|---|---|---|---|
| Amplitude (V) | 1000 | 1000 | 130 |
| Duration (ms) | 0.1 | 0.1 | 0.2 |
| Interval (ms) | 0.2 | 100 | 2 |
| Number | 1 | 1 | 4 |

In T-cells, three days post activation, activated T-cells were split into fresh complete media and cultured in fresh complete media overnight before the transfection/transduction on Day4 post activation. T-cells were transfected according to the following procedure. For TALEN mRNA transfection, cells were washed twice in Cytoporation buffer T (BTX Harvard Apparatus Cat #47-0002), and 5 million cells were then resuspended in 180 ml of Cytoporation buffer T. This cellular suspension was mixed with mRNA encoding TRAC TALEN at 0.5 μg mRNA per TALEN arm per million cells. Transfection was performed using Pulse Agile technology in 0.4 cm gap cuvettes ((#45-0126 BTX Harvard Apparatus) according to the program shown in Table 7.

TABLE 7
BTX PulseAgile settings for T cells

| Settings | Group1 | Group2 | Group3 |
|---|---|---|---|
| Amplitude (V) | 800 | 800 | 130 |
| Duration (ms) | 0.1 | 0.1 | 0.2 |
| Interval (ms) | 0.2 | 100 | 2 |
| Number | 1 | 1 | 4 |

### AAV Preparation and Transduction:

For HSCs, AAV-HLA-E particles was thawed on ice and aliquot to transduce each of the HSC samples at 10⁴ viral genome per cell (vg/cell). The chloroquine or hydroxychloroquine were mixed to AAV6 particles and left at RT for 5 mins before added to the cell culture. The amount of chloroquine or hydroxychloroquine used lead to the indicated final concentration in culture media. The transfected cells, together AAV6 and chloroquine or hydroxychloroquine mixture were incubated at 37° C., 5% CO₂ for an hour. After the 1 hr incubation at 37° C., the cells were collected and spun down, the supernatant was removed, and the cells were resuspended into 500 ml fresh expansion media and incubated overnight at 30° C. Cells were then counted and diluted at 0.3×10 6 cells/ml in expansion media and cultivated at 37° C. until analysis.

For T-cells, the AAV6 encoding Mesothelin CAR (SEQ NO: 28), targeted to TRAC locus, was thawed on ice and made into 1.4 10⁵ vg/cell aliquots in a 48-well. Different amount of chloroquine (0.05 mM, and 0.1 mM final concentration in cell culture) were mixed to the AAV6. The AAV6 and chloroquine mixture was added to the transfected T-cells and incubated with the cells for 1 hr in 37° C. After an hour of incubation, the cells were collected and spun down. The supernatant was removed, and the cells were resuspended into 300 ml fresh X-vivo with 5% AB serum (Gemini Cat #H47Y00L) and 20 ng/ml IL2 (Miltenyi biotec Cat #130-097-743) and moved to 30° C. for overnight incubation. T-cells were then counted and passaged at 1 10⁶ cells/ml in complete growth media and kept at 37° C. until analysis.

### Flow Cytometry Analysis:

To detect HLA-E and B2M expression on HSCs cell surface, HSCs were harvested at days post electroporation/transduction, washed once in 2% FBS/PBS and stained with HLA-ABC-Vioblue antibody (Catalog #130-120-435, Miltenyi) and HLA-E-APC antibody (Catalog #130-117-402, Miltenyi) for 20 mins at 4° C. The staining solution was then washed off and the cells were resuspended in 4% PFA/PBS before analysis with MacsQuant (Miltenyi).

To detect mesothelin-CAR expression on T-cell cell surface, 12 days after cells were transfected and transduced, T-cells were harvested and washed in 2% FBS/PBS and stained an anti-mouse Biotin-F(ab)2 antibody (Jackson ImmunoResearch #115-065-072) for 30 mins at 4° C. After anti-F(ab)2 antibody staining, the cells were washed twice in 2% FBS/PBS and stained with a APC-Streptavidin (Biolegen #405207). The cells were then stained with Anti-TCRa/b-PE (Miltenyi #130-113-539) to measure the TCRalpha on cell surface. The stained the cells were then fixed with 4% PFA/PBS before analyzed on BD Canto.

The main polynucleotide and polypeptide sequences referred to in these examples can be found in the sequence listing and in Tables 8 and 9.

### Example 2: Chloroquine Stimulates Nuclease Induced Targeted Integration of a DNA Repair Template in HSCs

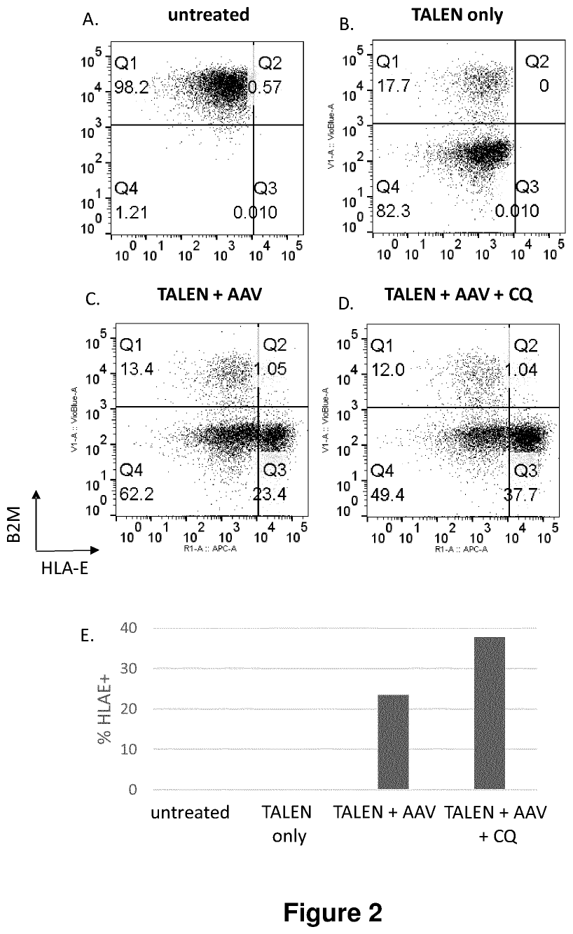

HSCs were transfected with TALEN and transduced with AAV-HLA-E repair template as indicated in example 1 with or without chloroquine at a final concentration of 0.1 mM in culture media. Expression of B2M (measured by HLA-ABC staining) reveals that, 5 days post transfection/transduction, B2M TALEN treatment, B2M TALEN with HLA-E repair matrix treatment and B2M TALEN with HLA-E repair matrix treatment in presence of chloroquine led to 82.3%, 85.6% and 87.7% (addition of cells present in the lower left and right panels) of B2M inhibition, respectively. This result demonstrates that Chloroquine increases B2M inactivation. 30 Most importantly, B2M TALEN with HLA-E repair matrix and B2M TALEN with HLA-E repair matrix treatment in presence of chloroquine showed 23.4% and almost of 38% of HLA-E expression (FIG. 2). Since HLA-E expression reflects targeted integration, these results demonstrate that chloroquine increases the level of targeted integration of HLA-E at the B2M locus induced by a B2M site specific nuclease.

### Example 3: Chloroquine Stimulates Nuclease-Induced Targeted Integration in Primary T-Cells

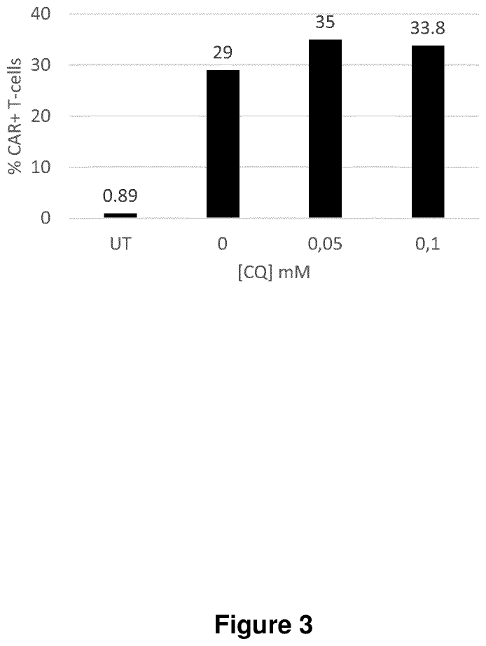

T-cells were transfected with TRAC TALEN and transduced with AAV-CAR-MESO repair template as indicated in example 1 with or without chloroquine at a final concentration of 0.05 or 0.1 mM. Since CAR expression was dependent on its integration the percentage of CAR positive cells reflects the nuclease-induced targeted integration. Results in FIG. 3 shows that without chloroquine targeted integration reached 29% whereas adding 0.05 mM and of chloroquine stimulates the nuclease-induced targeted integration up to 35 and 33.8% respectively. This result demonstrates that chloroquine can also stimulate nuclease-induced targeted integration at the TRAC locus in primary T-cells.

### Example 4: All Chloroquine Concentrations Stimulate Nuclease-Induced Targeted Integration

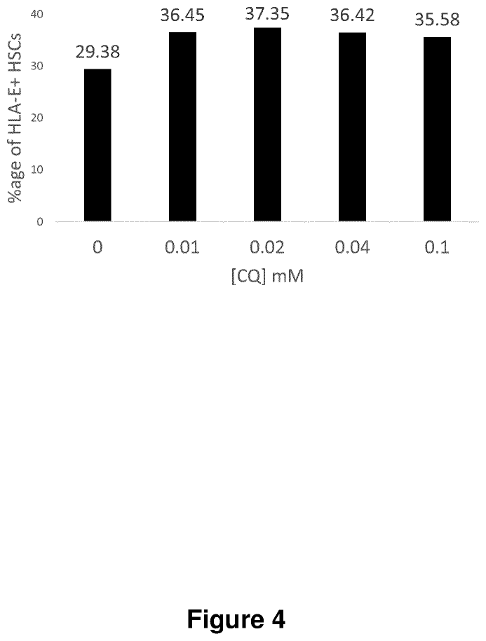

HSCs were transfected with TALEN and transduced with AAV-HLA-E repair template as indicated in example 1 with or without chloroquine (CQ) at the indicated final concentration varying from 0 to 0.1 mM. B2M TALEN with HLA-E repair matrix without chloroquine showed 29.4% of HLA-E positive HSCs, whereas all tested CQ concentrations showed an increase of HLA-E positive cells percentage up to 35.5% to 37.4% (FIG. 4) with an optimum CQ dose at 0.02 nM.

### Example 5: Hydroxychloroquine Improves Nuclease-Induced Targeted Integration

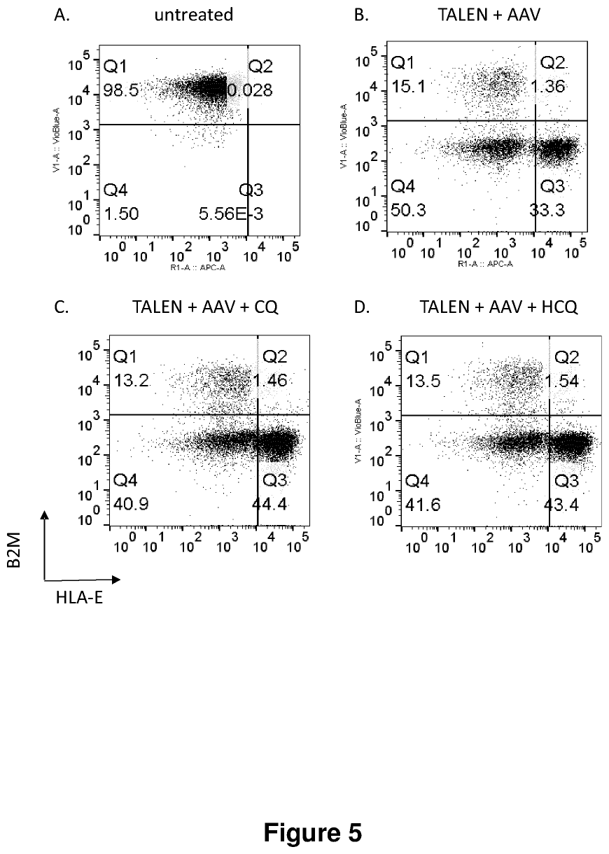

HSCs were transfected with B2M TALEN and transduced with AAV-HLA-E repair template as indicated in example 1 without or with chloroquine or hydroxychloroquine at a final concentration of 0.02 mM. Expression of B2M reveals that, 5 days post transfection/transduction, B2M TALEN with HLA-E repair matrix treatment and B2M TALEN with HLA-E repair matrix treatment in presence of chloroquine or hydroxychloroquine led to 83.6%, 85.3% and 85% (addition of cells present in the lower left and right panels) of B2M inhibition, respectively. The percentage of HLA-E positive cells increase from 33.3% without any compound up to 44.4% and 43.4% with chloroquine or hydroxychloroquine, respectively (FIG. 5). These results demonstrate that chloroquine as well as its derivative hydroxychloroquine are both able to increase the level of nuclease-induced targeted integration.

### Example 6: Chloroquine Potentiates Known Nuclease-Induced Targeted Integration Stimulators

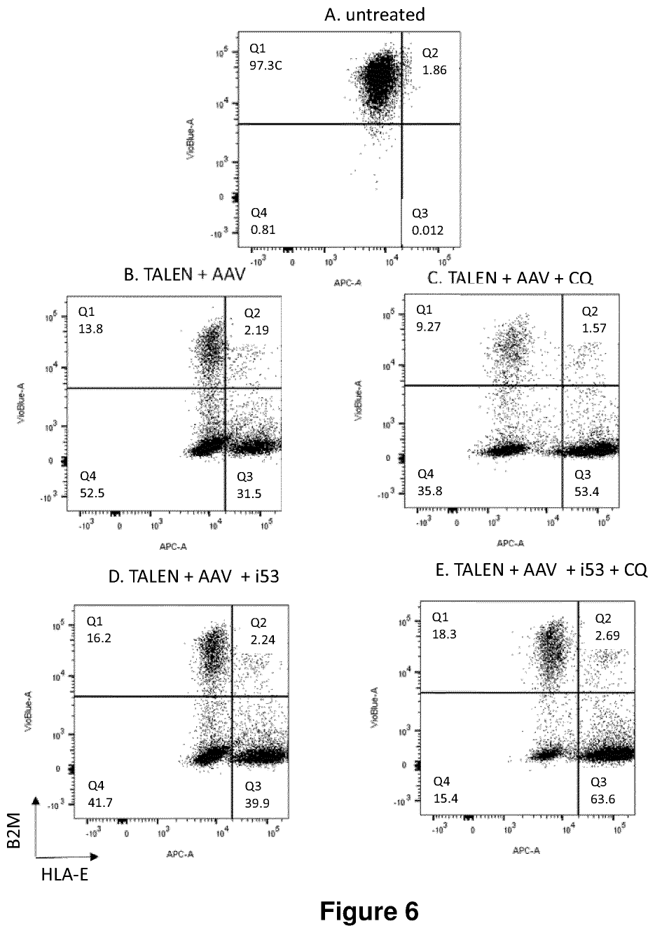

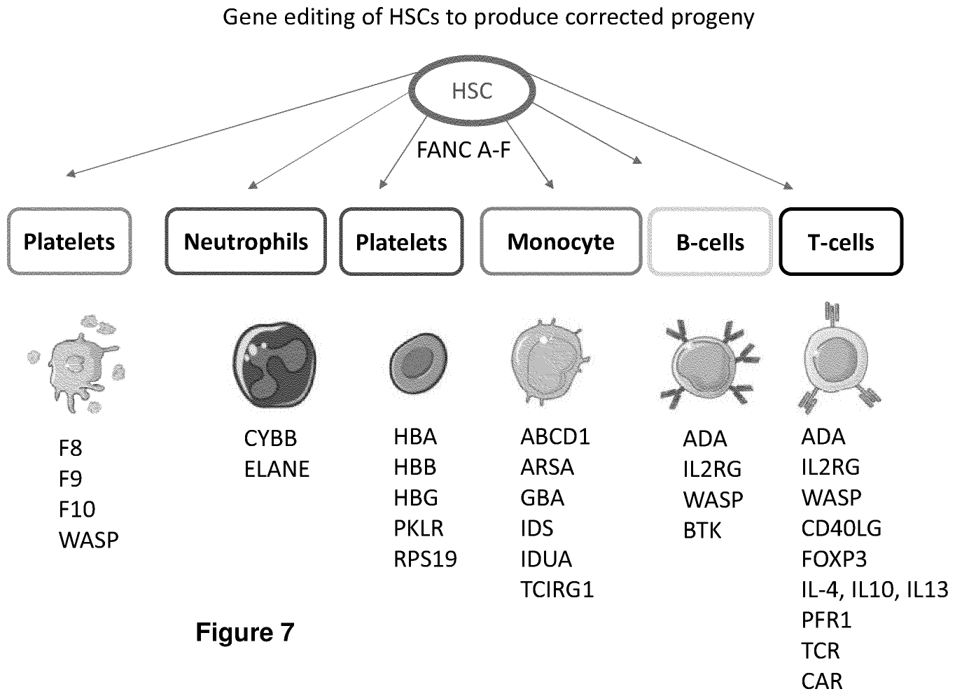

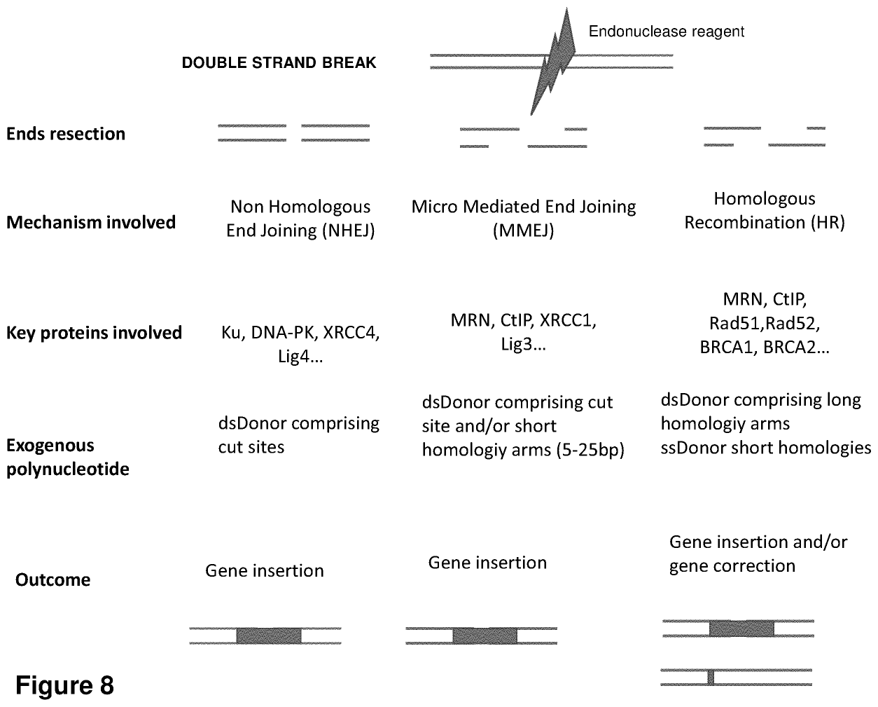

Stimulation of nuclease-induced targeted integration could be potentiated by expressing an 53BP1 inhibitor as described by Canny M. D., et al. [Inhibition of 53BP1 favors homology-dependent DNA repair and increases CRISPR-Cas9 genome-editing efficiency (2017) Nature Biotechnology, 36:95-102]. HSCs were thus transfected with B2M TALEN alone or with 4 μg/million cells mRNAs encoding i53 (SEQ ID NO. 40), respectively. HSCs were then transduced with AAV-HLA-E repair template as indicated in example 1 with or without chloroquine to a final concentration of 0.02 mM in culture media. Without 53BP1inhibition, the percentage of HLA-E positive cells increase from 31.5% without any compound to 53.4% in presence of CQ (FIGS. 6B and 6C). Inhibition of 53BP1 stimulates the percentage of HLA-E positive cells from 31.5% to 39.9% (FIGS. 6B and 6D). And most importantly, inhibition of 53BP1 combined with chloroquine led to the highest percentage of HLA-E positive cells: up to 63.6% (FIG. 6E). These results demonstrate that chloroquine can further potentiate the increase of nuclease-induced targeted integration by known factors, such as i53.

TABLE 8
Polynucleotide sequences used in the gene targeted integration experiments

| | SEQUENCE | |
|---|---|---|
| SEQ ID NO: # | designation | POLYNUCLEOTICE SEQUENCE |
| 27 | HLA-E AAV | CACCCCAGATCGGAGGGCGCCGATGTACAGACAGCAAACTCACC |
| | insert | CAGTCTAGTGCATGCCTTCTTAAACATCACGAGACTCTAAGAAAA |
| | | GGAAACTGAAAACGGGAAAGTCCCTCTCTCTAACCTGGCACTGC |
| | | GTCGCTGGCTTGGAGACAGGTGACGGTCCCTGCGGGCCTTGTCC |
| | | TGATTGGCTGGGCACGCGTTTAATATAAGTGGAGGCGTCGCGCT |
| | | GGCGGGCATTCCTGAAGCTGACAGCATTCGGGCCGAGATGTCTC |
| | | GCTCCGTGGCCTTAGCTGTGCTCGCGCTACTCTCTCTTAGCGGC |
| | | CTCGAAGCTGTTATGGCTCCGCGGACTTTAATTTTAGGTGGTGGC |
| | | GGATCCGGTGGTGGCGGTTCTGGTGGTGGCGGCTCCATCCAGC |
| | | GTACGCCCAAAATTCAAGTCTACAGCCGACATCCTGCAGAGAACG |
| | | GCAAATCTAATTTCCTGAACTGCTATGTATCAGGCTTTCACCCTAG |
| | | CGATATAGAAGTGGACCTGCTGAAAAACGGAGAGAGGATAGAAA |
| | | AGGTCGAACACAGCGACCTCTCCTTTTCCAAGGACTGGAGCTTTT |
| | | ATCTTCTGTATTATACTGAATTTACACCCACGGAAAAAGATGAGTA |
| | | TGCGTGCCGAGTAAACCACGTCACGCTGTCACAGCCCAAAATAGT |
| | | AAAATGGGATCGCGACATGGGTGGTGGCGGTTCTGGTGGTGGCG |
| | | GTAGTGGCGGCGGAGGAAGCGGTGGTGGCGGTTCCGGATCTCA |
| | | CTCCTTGAAGTATTTCCACACTTCCGTGTCCCGGCCCGGCCGCG |
| | | GGGAGCCCCGCTTCATCTCTGTGGGCTACGTGGACGACACCCAG |
| | | TTCGTGCGCTTCGACAACGACGCCGCGAGTCCGAGGATGGTGCC |
| | | GCGGGCGCCGTGGATGGAGCAGGAGGGGTCAGAGTATTGGGAC |
| | | CGGGAGACACGGAGCGCCAGGGACACCGCACAGATTTTCCGAGT |
| | | GAACCTGCGGACGCTGCGCGGCTACTACAATCAGAGCGAGGCC |
| | | GGGTCTCACACCCTGCAGTGGATGCATGGCTGCGAGCTGGGGC |
| | | CCGACAGGCGCTTCCTCCGCGGGTATGAACAGTTCGCCTACGAC |
| | | GGCAAGGATTATCTCACCCTGAATGAGGACCTGCGCTCCTGGAC |
| | | CGCGGTGGACACGGCGGCTCAGATCTCCGAGCAAAAGTCAAATG |
| | | ATGCCTCTGAGGCGGAGCACCAGAGAGCCTACCTGGAAGACACA |
| | | TGCGTGGAGTGGCTCCACAAATACCTGGAGAAGGGGAAGGAGAC |
| | | GCTGCTTCACCTGGAGCCCCCAAAGACACACGTGACTCACCACC |
| | | CCATCTCTGACCATGAGGCCACCCTGAGGTGCTGGGCTCTGGGC |
| | | TTCTACCCTGCGGAGATCACACTGACCTGGCAGCAGGATGGGGA |
| | | GGGCCATACCCAGGACACGGAGCTCGTGGAGACCAGGCCTGCA |
| | | GGGGATGGAACCTTCCAGAAGTGGGCAGCTGTGGTGGTGCCTTC |
| | | TGGAGAGGAGCAGAGATACACGTGCCATGTGCAGCATGAGGGGC |
| | | TACCCGAGCCCGTCACCCTGAGATGGAAGCCGGCTTCCCAGCCC |
| | | ACCATCCCCATCGTGGGCATCATTGCTGGCCTGGTTCTCCTTGGA |
| | | TCTGTGGTCTCTGGAGCTGTGGTTGCTGCTGTGATATGGAGGAA |
| | | GAAGAGCTCAGGTGGAAAAGGAGGGAGCTACTATAAGGCTGAGT |
| | | GGAGCGACAGTGCCCAGGGGTCTGAGTCTCACAGCTTGTAACTG |
| | | TGCCTTCTAGTTGCCAGCCATCTGTTGTTTGCCCCTCCCCCGTGC |
| | | CTTCCTTGACCCTGGAAGGTGCCACTCCCACTGTCCTTTCCTAAT |
| | | AAAATGAGGAAATTGCATCGCATTGTCTGAGTAGGTGTCATTCTAT |
| | | TCTGGGGGGTGGGGTGGGGCAGGACAGCAAGGGGGAGGATTGG |
| | | GAAGACAATAGCAGGCATGCTGGGGATGCGGTGGGCTCTATGTC |
| | | TCTTTCTGGCCTGGAGGCTATCCAGCGTGAGTCTCTCCTACCCTC |
| | | CCGCTCTGGTCCTTCCTCTCCCGCTCTGCACCCTCTGTGGCCCTC |
| | | GCTGTGCTCTCTCGCTCCGTGACTTCCCTTCTCCAAGTTCTCCTT |
| | | GGTGGCCCGCCGTGGGGCTAGTCCAGGGCTGGATCTCGGGGAA |
| | | GCGGCGGGGTGGCCTGGGAGTGGGGAAGGGGGTGCGCACCCG |
| | | GGACGCGCGCTACTTGCCCCTTTCGGCGGGGAGCAGGGGAGAC |
| | | CTTTGGCCTACGGCGACGGGAGGGTCGGGACA |
| 28 | Mesothelin | GATAAGTAGCCCTGCATTTCAGGTTTCCTTGAGTGGCAGGCCAGG |
| | CAR AAV | CCTGGCCGTGAACGTTCACTGAAATCATGGCCTCTTGGCCAAGAT |
| | insert | TGATAGCTTGTGCCTGTCCCTGAGTCCCAGTCCATCACGAGCAGC |
| | | TGGTTTCTAAGATGCTATTTCCCGTATAAAGCATGAGACCGTGACT |
| | | TGCCAGCCCCACAGAGCCCCGCCCTTGTCCATCACTGGCATCTG |
| | | GACTCCAGCCTGGGTTGGGGCAAAGAGGGAAATGAGATCATGTC |
| | | CTAACCCTGATCCTCTTGTCCCACAGATATCCAGGGCAGCGGCGT |
| | | GAAGCAGACCCTGAACTTCGACCTGCTGAAGCTGGCAGGCGATG |
| | | TGGAGTCCAATCCAGGACCTATGGCTCTGCCCGTCACCGCTCTG |
| | | CTGCTGCCACTGGCCCTGCTGCTGCACGCAGCAAGGCCACAGGT |
| | | GCAGCTGCAGCAGCCTGGCGCAGAGCTGGTGAAGCCTGGCGCC |
| | | AGCATGAAGCTGTCCTGCAAGGCCTCTGGCTACACATTCACCTCC |
| | | TATTGGATGCACTGGGTGAAGCAGCGCCCAGGCCAGGGACTGGA |
| | | GTGGATCGGCATGATCCACCCCAACTCTGACAATACCATCTACTA |
| | | TGAGAAGTTTAAGAGCAAGGCCACACTGACCGTGGATAAGAGCT |
| | | CCTCTACAGCCTACATGCAGCTGAGCTCCCTGACCTCCGAGGAC |
| | | TCTGCCGTGTACTATTGCGCCATCATCATCACACCCGTGGTGCCT |
| | | AAGTTCGATTATTGGGGCCAGGGCACCACACTGACCGTGTCTAG |
| | | CGGAGGAGGAGGAAGCGGAGGAGGAGAATCCGGCGGCGGCGG |
| | | CTCTGACATCGTGATGACACAGAGCCACCAGTTTATGAGCACCTC |
| | | CGTGGGCGACCGGGTGAGCGTGACCTGTAAGGCCTCCCACGAT |
| | | GTGGGCACCTCTGTGGCCTGGTACCAGCAGAAGCCAGGCCAGA |
| | | GCCCCAAGCTGCTGATCTATTGGGCCTCCACAAGGCACACCGGA |
| | | GTGCCAGACCGCTTCACAGGATCTGGAAGCGGCACCGACTTCAC |
| | | CCTGACCATCAGCAACGTGCAGTCCGAGGACCTGGCCGATTACT |
| | | TCTGTCAGCAGTACTCCTCTTATCCTCTGACATTTGGCGCAGGAA |
| | | CCAAGCTGGAGCTGAAGAGGGCCTCTGATCCAGGCTCCGGCGG |
| | | AGGAGAATCCTGCCCTTACAGCAACCCATCCCTGTGCTCTGGAG |
| | | GAGGAGGATCTTGTCCCTATAGCAATCCTAGCCTGTGCTCCGGC |
| | | GGAGGAGGCAGCACCACAACCCCAGCACCAAGGCCACCTACACC |
| | | TGCACCAACCATCGCATCCCAGCCACTGTCTCTGAGGCCAGAGG |
| | | CATGCAGACCTGCAGCAGGCGGCGCCGTGCACACCAGAGGCCT |
| | | GGACTTCGCCTGCGATATCTACATCTGGGCACCTCTGGCAGGAA |
| | | CATGTGGCGTGCTGCTGCTGTCCCTGGTCATCACCCTGTATTGTA |
| | | AGCGAGGCCGGAAGAAACTGCTGTATATTTTCAAACAGCCCTTTA |
| | | TGAGACCTGTGCAGACTACCCAGGAGGAAGACGGCTGCAGCTGT |
| | | AGGTTCCCCGAGGAAGAGGAAGGCGGGTGTGAGCTGAGGGTCA |
| | | AGTTTAGCCGCTCCGCAGATGCCCCTGCTTACCAGCAGGGGCAG |
| | | AATCAGCTGTATAACGAGCTGAATCTGGGACGGAGAGAGGAATA |
| | | CGACGTGCTGGATAAAAGGCGCGGGAGAGACCCCGAAATGGGA |
| | | GGCAAGCCACGACGGAAAAACCCCCAGGAGGGCCTGTACAATGA |
| | | ACTGCAGAAGGACAAAATGGCAGAGGCCTATAGTGAAATCGGGA |
| | | TGAAGGGAGAGAGAAGGCGCGGCAAAGGGCACGATGGCCTGTA |
| | | CCAGGGGCTGTCTACTGCCACCAAGGACACCTATGATGCTCTGC |
| | | ATATGCAGGCACTGCCTCCAAGGTGATAATCTAGAGGGCCCGTTT |
| | | AAACCCGCTGATCAGCCTCGACTGTGCCTTCTAGTTGCCAGCCAT |
| | | CTGTTGTTTGCCCCTCCCCCGTGCCTTCCTTGACCCTGGAAGGTG |
| | | CCACTCCCACTGTCCTTTCCTAATAAAATGAGGAAATTGCATCGCA |
| | | TTGTCTGAGTAGGTGTCATTCTATTCTGGGGGGTGGGGTGGGGC |
| | | AGGACAGCAAGGGGGAGGATTGGGAAGACAATAGCAGGCATGCT |
| | | GGGGATGCGGTGGGCTCTATGACTAGTGGCGAATTCCCGTGTAC |
| | | CAGCTGAGAGACTCTAAATCCAGTGACAAGTCTGTCTGCCTATTC |
| | | ACCGATTTTGATTCTCAAACAAATGTGTCACAAAGTAAGGATTCTG |
| | | ATGTGTATATCACAGACAAAACTGTGCTAGACATGAGGTCTATGG |
| | | ACTTCAAGAGCAACAGTGCTGTGGCCTGGAGCAACAAATCTGACT |
| | | TTGCATGTGCAAACGCCTTCAACAACAGCATTATTCCAGAAGACA |
| | | CCTTCTTCCCCAGCCCAGGTAAGGGCAGCTTTGGTGCCTTCGCA |
| | | GGCTGTTTCCTTGCTTCAGGAAATCGGATCCCCCAGGTAGATAAG |
| | | TAGCA |
| 29 | BGH poly A | CTGTGCCTTCTAGTTGCCAGCCATCTGTTGTTTGCCCCTCCCCCG |
| | sequence | TGCCTTCCTTGACCCTGGAAGGTGCCACTCCCACTGTCCTTTCCT |
| | | AATAAAATGAGGAAATTGCATCGCATTGTCTGAGTAGGTGTCATTC |
| | | TATTCTGGGGGGTGGGGTGGGGCAGGACAGCAAGGGGGAGGAT |
| | | TGGGAAGACAATAGCAGGCATGCTGGGGATGCGGTGGGCTCTATG |
| 30 | B2M Left | CGCGCACCCCAGATCGGAGGGCGCCGATGTACAGACAGCAAACT |
| | homology arm | CACCCAGTCTAGTGCATGCCTTCTTAAACATCACGAGACTCTAAG |
| | | AAAAGGAAACTGAAAACGGGAAAGTCCCTCTCTCTAACCTGGCAC |
| | | TGCGTCGCTGGCTTGGAGACAGGTGACGGTCCCTGCGGGCCTTG |
| | | TCCTGATTGGCTGGGCACGCGTTTAATATAAGTGGAGGCGTCGC |
| | | GCTGGCGGGCATTCCTGAAGCTGACAGCATTCGGGCCGAGATGT |
| | | CTCGCTCCGTGGCCTTAGCTGTGCTCGCGCTACTC |
| 31 | B2M Right | TCTCTTTCTGGCCTGGAGGCTATCCAGCGTGAGTCTCTCCTACCC |
| | homology arm | TCCCGCTCTGGTCCTTCCTCTCCCGCTCTGCACCCTCTGTGGCCC |
| | | TCGCTGTGCTCTCTCGCTCCGTGACTTCCCTTCTCCAAGTTCTCC |
| | | TTGGTGGCCCGCCGTGGGGCTAGTCCAGGGCTGGATCTCGGGG |
| | | AAGCGGCGGGGTGGCCTGGGAGTGGGGAAGGGGGTGCGCACC |
| | | CGGGACGCGCGCTACTTGCCCCTTTCGGCGGGGAGCAGGGGAG |
| | | ACCTTTGGCCTACGGCGACGGGAGGGTCGGGACAAAG |

TABLE 9
Polypeptide sequences used in the gene targeted integration experiments

| | SEQUENCE | |
|---|---|---|
| SEQ ID NO: # | designation | POLYNUCLEOTIDE SEQUENCE |
| 40 | i53 Prot. | MLIFVKTLTGKTITLEVEPSDTIENVKAKIQDKEGIPPDQQRLAFAGKSLEDG |
| | Seq | RTLSDYNILKDSKLHPLLRLR |
| 41 | Bclxl | MSQSNRELVVDFLSYKLSQKGYSWSQFSDVEENRTEAPEGTESEMETPSAING |
| | Prot. Seq | NPSWHLADSPAVNGATGHSSSLDAREVIPMAAVKQALREAGDEFELRYRRAFS |
| | | DLTSQLHITPGTAYQSFEQVVNELFRDGVNWGRIVAFFSFGGALCVESVDKEM |
| | | QVLVSRIAAWMATYLNDHLEPWIQENGGWDTFVELYGNNAAAESRKGQERFNR |
| | | WFLTGMTVAGVVLLGSLFSRK |
| 42 | B2M Signal | SLSGLEA |
| | Seq | |
| 43 | HLA-G | VMAPRTLIL |
| | peptid | |
| 44 | 3xG4S | GGGGSGGGGSGGGGS |
| | linker | |
| 45 | B2M | IQRTPKIQVYSRHPAENGKSNFLNCYVSGFHPSDIEVDLLKNGERIEKVEHSD |
| | peptide | LSFSKDWSFYLLYYTEFTPTEKDEYACRVNHVTLSQPKIVKWDRDM |
| 46 | 4xG4S | GGGGSGGGGSGGGGSGGGGS |
| | linker | |
| 47 | HLA-E full | HSLKYFHTSVSRPGRGEPRFISVGYVDDTQFVRFDNDAASPRMVPRAPWMEQE |
| | length | GSEYWDRETRSARDTAQIFRVNLRTLRGYYNQSEAGSHTLQWMHGCELGPDRR |
| | | FLRGYEQFAYDGKDYLTLNEDLRSWTAVDTAAQISEQKSNDASEAEHQRAYLE |
| | | DTCVEWLHKYLEKGKETLLHLEPPKTHVTHHPISDHEATLRCWALGFYPAEIT |
| | | LTWQQDGEGHTQDTELVETRPAGDGTFQKWAAVVVPSGEEQRYTCHVQHEGLP |
| | | EPVTLRWKPASQPTIPIVGIIAGLVLLGSVVSGAVVAAVIWRKKSSGGKGGSY |
| | | YKAEWSDSAQGSESHSL |

1) Use of aminoquinoline compound(s) to increase the frequency of targeted genome modification by a sequence-specific gene editing reagent at a selected locus in the genome of a cell.

2) Use according to claim 1, wherein said targeted genome modification is the targeted integration at said locus of an exogenous nucleic acid template.

3) Use according to any one of claim 1 or 2, wherein said gene editing reagent is a sequence-specific endonuclease or nickase reagent.

4) Method to increase the frequency of targeted modification into the genome of a cell, characterized in that said method comprises the step of treating the cell with a sequence-specific endonuclease or nickase reagent and at least one aminoquinoline compound(s).

5) Method according to claim 4, comprising the step of introducing into the cell an exogenous nucleic acid template to be integrated at the locus targeted by said sequence-specific nuclease or nickase reagent.

6) Method for targeted integration of an exogenous nucleic acid template at a selected locus in the genome of cells, said method comprising the steps of:
i) contacting the cells with aminoquinoline compound(s);
ii) introducing into said cells at least one sequence-specific endonuclease or nickase reagent that specifically targets said selected locus,
iii) introducing into said cells an exogenous nucleic acid template to be integrated at said locus,
iv) cultivating the cells to induce DNA repair and integration of the exogenous nucleic acid template at said selected locus targeted by said sequence-specific endonuclease or nickase;
v) optionally, selecting the cells which have integrated the exogenous nucleic acid template at the selected locus in their genome.

7) Use or method according to any one of claims 3 to 6, wherein said introduction of said sequence-specific nickase or endonuclease reagent into the cells is performed by electroporation.

8) Use or method according to any one of claims 2 to 7, wherein said exogenous nucleic acid template to be integrated at said locus is comprised into a non-integrative viral vector such as an IDLV or AAV.

9) Use or method according to any one of claims 2 to 8, wherein said exogenous nucleic acid template integration at said selected locus is obtained by homologous recombination.

10) Use or method according to any one of claims 1 to 9, wherein said aminoquinoline compound is a derivative of 4-aminoquinoline or 8-aminoquinoline.

11) Use or method according to any one of claims 1 to 10, wherein said aminoquinoline compound is choloroquine, chloroquine phosphate, hydroxychloroquine, chloroquine diphosphate, chloroquine sulphate, hydroxychloroquine sulphate, or enantiomers, derivatives, analogs, metabolites, pharmaceutically acceptable salts, and mixtures thereof.

12) Use or method according to any one of claims 1 to 11, wherein said aminoquinoline compound is selected from 7-chloro-4-(4-diethylamino-1-butylamino)quinoline (desmethylchloroquine); 7-hydroxy-4-(4-diethylamino-1-butylamino)quinoline; 7-chloro-4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-hydroxy-4-(1-carboxy-4-diethylamino-1-methylbutylamino)quinoline; 7-chloro-4-(4-ethyl-(2-hydroxyethyl)-amino-1-methylbutylamino)quinoline (hydroxychloroquine); 7-hydroxy-4-(4-ethyl-(2-hydroxyethyl)-amino-1-methyl-1-butylamino)quinoline; hydroxychloroquine phosphate; 7-chloro-4-(4-ethyl-(2-hydroxyethyl)-amino-1-butylamino)quinoline (desmethylhydroxychloroquine); 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-hydroxy-4-(1-carboxy-4-ethyl-(-2-hydroxyethyl)-amino-1-methylbutylamino)quinoline; 8-[(4-aminopentyl)amino]-6-methoxydihydrochloride quinoline; 1-acetyl-1,2,3,4-tetrahydroquinoline; 8-[4-aminopentyl)amino]-6-methoxyquinoline dihydrochloride; 1-butyryl-1,2,3,4-tetrahydroquinoline; 7-chloro-2-(o-chlorostyryl)-4-[4-diethylamino-1-methylbutyl]aminoquinoline phosphate; 3-chloro-4-(4-hydroxy-.alpha.,.alpha.′-bis(2-methyl-1-pyrrolidinyl)-2,5-xylidinoquinoline, 4-[(4-diethylamino)-1-methylbutyl)amino]-6-methoxyquinoline; 3,4-dihydro-1(2H)-quinolinecarboxyaldehyde; 1,1′-pentamethylenediquinoleinium diiodide; and 8-quinolinol sulfate, enantiomers thereof, as well as suitable pharmaceutical salts thereof.

13) Use or method according to any one of claims 1 to 12, wherein said cell is a eucaryotic cell, preferably a plant or animal cell, preferably a primate cell, preferably a human cell.

14) Use or method according to any one of claims 1 to 13, wherein said cell is a primary eucaryotic cell.

15) Use or method according to any one of claims 1 to 14, wherein said cell is a pluripotent stem cell, preferably iPS or ES cells.

16) Use or method according to any one of claims 1 to 15, wherein said cell is a hematopoietic stem cell (HSC).

17) Use or method according to any one of claims 1 to 16, wherein said selected locus is chosen from the group consisting of: CCR5, HBB, AAVS1, STAT3, ADPS1, RAG1, TMEM119, MERTK, CD164, TLR7, CD14, FCGR3A (CD16), TBXAS1, DOK3, ABCA1, TMEM195, TLR4, MR1, FCGR1A (CD64), CSF3R, FGD4, TSPAN14, CXCR3, CD11B, S100A9, B2M. IL2RG, ADA, WAS, Gp91phox, CD18, DCLRE1C, FANCA, ARSA, ABCD1 and IDUA.

18) Use or method according to any one of claims 1 to 14, wherein said cell is a white blood cell, such as macrophage, a dendritic cell, a lymphocyte, preferably a T-cell or NK-cell.

19) Use or method according to any one of claims 1 to 18, wherein said selected locus in said cell is chosen from the group consisting of: TCR, GM-CSF, B2M, GCN2, PD1, CTLA4, TIM3, LAG3, DCK, HPRT, GGH, GR, CD52, TGFb, TGFbR (TGFbeta receptor) IL-10, IL-10R or CISH.

20) Use or method according to any one of claims 1 to 18, wherein said selected locus in said cell is chosen from the group consisting of: TGF-β receptor, Cbl-B, A2A receptor, KLRD1, LIR1/ILT2, KIRs, AhR, Tim-3, Tyro-3, GCN2, CD94, CD74, cyclophilin A, TBL1XR1, HPRT, dCK, CD5, beta2M and PD-1.

21) Use or method according to any one of claims 3 to 20, wherein said sequence specific endonuclease is a rare-cutting endonuclease, such as a RNA-guided endonuclease, such as CRISPR, RNA guide nickase, such as Cas9n, TALE-nuclease, such as TALEN or mega-TALE ZFN or meganucleases, such as engineered homing endonucleases.

22) Use or method according to any one of claims 2 to 21, wherein said exogenous nucleic acid template is provided as a plasmid.

23) Use or method according to any one of claims 2 to 21, wherein said exogenous nucleic acid template is double stranded (dsDNA), such as a PCR product.

24) Use or method according to claim 23, wherein said dsDNA has a length of more than 2 kb, preferably more than 2.5 kb, more preferably more than 3 kb, even more preferably between 2 and 10 kb.

25) Use or method according to any one of claims 2 to 21, wherein said nucleic acid template is a single stranded polynucleotide, such as a short single-stranded oligodeoxynucleotide (ssODN).

26) Use or method according to any one of claims 2 to 25, wherein said exogenous nucleic acid template is transfected in the cell by electroporation.

27) Use or method according to any one of claims 2 to 26, wherein said aminoquinoline compound is mixed with the nucleic acid template in the transduction buffer.

28) Use or method according to any one of claims 1 to 27, wherein said aminoquinoline compound is included into nanoparticles, such as silica based mesoporous particles.

29) Use or method according to any one of claims 2 to 28, wherein said exogenous nucleic acid template comprises the partial or complete nucleic acid sequence transgene selected from a chimeric antigen receptor (CAR), HLAE, HLAG, HBB, STAT3, ADPS1, RAG1, IL2RG, ADA, WAS, Gp91phox, CD18, DCLRE1C, FANCA, ARSA, ABCD1, IDUA, IDS, ARSB, GUSB, ABCD1, GALC, ARSA, PSAP, GBA, FUCA1, MAN2B1, AGA, ASAH1, HEXA, GAA, SMPD1, LIPA, CDKL5, ALDH, MGMT, MTX, GST, cytidine deaminase, IL2 receptor (CD25), IL15-2A-IL15 receptor, IFN gamma, Lysteria P60, TNF and IL12-α.

30) Use or method according to any one of claims 2 to 29, wherein said exogenous nucleic acid template comprises a corrected sequence to perform gene repair at said selected locus.

31) Use or method according to claim 30, wherein said selected locus is chosen from the group consisting of: HBB, IL2RG, ADA, WAS, Gp91phox, CD18, DCLRE1C, FANCA, ARSA, ABCD1, IDUA, IDS, ARSB, GUSB, ABCD1, GALC, ARSA, PSAP, GBA, FUCA1, MAN2B1, AGA, ASAH1, HEXA, GAA, SMPD1, LIPA and CDKL5.

32) Use or method according to any one of claims 4 to 31, wherein the cells are further treated with at least one compound selected from: STL127705, NU7441, KU-0060648, NU7026, M3812, E-822, SCR7, RS-1, Wortmanin, Aphidicolin, mimosin thymidine, Hydroxy urea (HU), Nocodazole, ABT-751, XL413, L755507, Brefeldin and Resveratrol to increase induced targeted integration.

33) Use or method according to any one of claims 4 to 32, wherein the cells are further treated with at least one inhibitor of lig4, xrcc4, Ku70, Ku80, DNA-PKcs, preferably shRNA or siRNA.

34) Use or method according to any one of claims 4 to 33, further comprising expressing into the cells a nucleic acid encoding Rad51, Rad52, E4orf6/7, dominant-negative p53 mutant protein (GSE56), inhibitor of 53PB1 and/or dominant-negative 53BP1.

35) Use or method according to any one of claims 1 to 34, for use in gene therapy.

36) Use or method according to any one of claims 1 to 35, wherein said use or method is performed ex-vivo.

37) Use or method according to claim 36, wherein said method comprises a further step of infusing the cells that have integrated the nucleic acid template at said selected locus into an organism.

38) Use or method according to claim 36, wherein said method comprises a further step of infusing the cells that have integrated the nucleic acid template at said selected locus into a patient.

39) An oligo capture assay (OCA), characterized in that cells are treated with an aminoquinoline compound(s) to increase oligonucleotide markers integration into the genome of said cells.

40) A composition, therapeutic composition, kit, or nanoparticle comprising:
(a) an aminoquinoline compound, and
(b) an exogenous nucleic acid template to be integrated into the genome of a cell at a selected locus.

41) A composition, therapeutic composition, kit, or nanoparticle according to claim 40, further comprising: (c) a sequence specific gene editing endonuclease or nickase reagent.

42) A composition, therapeutic composition, kit, or nanoparticle according to claim 40 or 41, further comprising at least one compound to increase induced targeted integration selected from: STL127705, NU7441, KU-0060648, NU7026, M3812, E-822, SCR7, RS-1, Wortmanin, Aphidicolin, mimosin thymidine, Hydroxy urea (HU), Nocodazole, ABT-751, XL413, L755507, Brefeldin and Resveratrol.

43) A composition, therapeutic composition, kit, or nanoparticle according to any one of claims 40 to 42, further comprising at least one inhibitor of lig4, xrcc4, Ku70, Ku80, DNA-PKcs, preferably shRNA or siRNA.

44) A composition, therapeutic composition, kit, or nanoparticle according to any one of claims 40 to 43, further comprising a nucleic acid expressing Rad51, Rad52, E4orf6/7, dominant-negative p53 mutant protein (GSE56), inhibitor of 53PB1 and/or dominant-negative 53BP1.

45) A cell culture medium comprising at least 0.005 mM of an aminoquinoline compound (as claimed before), preferably between 0.01 and 0.5 mM.

46) A cell culture medium comprising between 0.005 and 1 mM, preferably between 0.01 and 0.5 mM, and more preferably between 0.01 and 0.1 mM chloroquine or hydroxychloroquine.

47) An ex-vivo gene therapy method comprising the step of contacting a cell sequentially or concomitantly with (1) an aminoquinoline compound, and (2) an exogenous nucleic acid template, and optionally (3) sequence-specific gene editing reagent, preferably a nuclease or nickase reagent.

48) A gene therapy method comprising the step of administrating, sequentially or in combination: (a) an aminoquinoline compound, and (b) an exogenous nucleic acid template, and optionally (c) a sequence-specific gene editing nuclease reagent.

49) A gene therapy method according to claim 47 or 48, wherein said method further comprises contacting the cell with at least one compound selected from: STL127705, NU7441, KU-0060648, NU7026, M3812, E-822, SCR7, RS-1, Wortmanin, Aphidicolin, mimosin thymidine, Hydroxy urea (HU), Nocodazole, ABT-751, XL413, L755507, Brefeldin and Resveratrol.

50) A gene therapy method according to any one of claims 47 to 49, wherein said method further comprises contacting the cell with at least one inhibitor of lig4, xrcc4, Ku70, Ku80, DNA-PKcs, preferably shRNA or siRNA.

51) A gene therapy method according to any one of claims 47 to 50, wherein said method further comprises expressing into the cell a nucleic acid expressing Rad51, Rad52, E4orf6/7, dominant-negative p53 mutant protein (GSE56), inhibitor of 53PB1 and/or dominant-negative 53BP1.
